(19) United States
(12) Patent Application Publication
(10) Pub. No.: US 2024/0285732 A1
(43) Pub. Date: Aug. 29, 2024

(54) Patient Profiling for Antigen-Specific Immunomodulatory Therapies

(71) Applicant: PHAIM PHARMA LTD., London (GB)

(72) Inventors: Tihamer ORBAN, Brookline, MA (US); Jalahej HEYMAN, London (GB); Nara DAUBENEY, London (GB); Piers DAUBENEY, London (GB)

(21) Appl. No.: 18/571,294

(22) PCT Filed: Jun. 16, 2022
(86) PCT No.: PCT/IB2022/055607

§ 371 Date: Dec. 18, 2023

Related U.S. Application Data
(60) Provisional application No. 63/211,752, filed on Jun. 17, 2021.

(51) Int. Cl. A61K 38/28; A61M 5/00; C12Q 1/6883; G01N 33/50; G01N 33/68

(52) U.S. Cl. CPC A61K 38/28; A61M 5/003; C12Q 1/6883; G01N 33/505; G01N 33/6866; C12Q 2600/158; G01N 2333/57; G01N 2800/042; G01N 2800/52

(57) ABSTRACT

Compositions of peptide fragments of preproinsulin for the treatment of type 1 diabetes and methods for selecting one or more peptide fragments of preproinsulin suitable for subject-specific immunomodulatory therapy for type 1 diabetes. The one or more peptides may be selected based on an autoimmune phenotype for the subject, which may be characterized by a stimulation assay and/or the based on a genotype for one or more genes related to type 1 diabetes.

### TECHNICAL FIELD OF THE INVENTION

The present disclosure relates generally to the field of autoimmune disease and specifically to the treatment, prevention, or delayed progression of type 1 diabetes mellitus (T1DM). The present disclosure relates more particularly to immunomodulatory therapy for (T1DM) autoimmunity.

### BACKGROUND OF THE INVENTION

The onset of human type 1 diabetes mellitus (T1DM) is the clinical manifestation of B-cell failure caused by T cell mediated autoimmune destruction. T1DM results in a lifelong dependence on daily insulin injections and exposure to both the acute and late complications. Despite the significant progress that has been made in its treatment, T1DM represents a severe burden on the individual and on society. T1DM is a particular burden to children and their families, representing one of the most severe chronic childhood diseases. While the onset of T1DM can occur in adulthood, it is largely a problem in children and youngsters. There is a bimodal peak age of T1DM onset, between ages 4-7 and ages 14-16 years. The worldwide incidence of T1DM is increasing, with the greatest increase in children under the age of 5 years. Therefore, there is an urgent and growing need to ameliorate this disease.

T1DM is a common endocrine disease in children, and up to 80% of children with T1DM also has diabetic ketoacidosis (DKA), which is associated with both short-term risks and long-term consequences. Short-term, and often life threatening, complications include hypo and hyperglycemic episodes often complicated with acidosis. Long-term complications can represent further significant morbidity and mortality. Patients may face both macro and microvascular complications, cardiovascular complications, hypertension, retinopathy, nephropathy, and neuropathy, which can be debilitating and life threatening. These can be reduced with improved care, but currently cannot be eliminated in T1DM patients. Further severe complications include kidney failure, blindness, and amputation.

Despite the significant progress that has been made in its treatment, autoimmune-associated diabetes places a severe burden on affected individuals as well as on society. Insulin-dependent T1DM is an autoimmune disease, in which insulitis leads to the destruction of pancreatic B-cells. At the time of clinical onset of T1DM, significant numbers of insulin producing B-cells are destroyed, leaving only about 15% to 40% still capable of insulin production (McCulloch et al., Diabetes, 40:673-679 (1991)). This β-cell failure results in a life-long dependence on daily insulin injections and development of acute and late complications of the disease. During the natural history of the disease, the remaining functional population of β-cells inevitably dies, rendering patients dependent on exogenous insulin for life. The arrest or even the slowing of further destruction of B-cells is thus an unmet need in the field, the accomplishment of which would lead to prolonged remission and delay diabetes-related complications.

### BRIEF SUMMARY OF THE INVENTION

The present disclosure, in various aspects and embodiments, provides immunomodulatory therapy for type 1 diabetes mellitus (T1DM), including therapeutics, therapies, diagnostics, kits, and methods for making the same. For example, the disclosure provides compositions comprising a therapeutically effective amount of one or more peptide fragments of preproinsulin. The compositions can be used for treating T1DM. In addition to being immunomodulatory (e.g., as opposed to immunosuppressive), certain therapeutics in accordance with the present disclosure are not metabolically active (e.g., without insulin-like activity) and are, thus, advantageously safe for use (i.e., a large dose would not kill or harm a patient, as might a large dose of insulin).

The present disclosure, in various aspects and embodiments, provides methods for selecting one or more peptide fragments of preproinsulin for the treatment of a subject and/or methods of testing a subject's immune response or predicted immune response to one or more peptide fragments of preproinsulin. According to some aspects, compositions of select peptides are configured for treating individual subjects and/or for treating select populations of subjects. The selection of peptide fragments may be based on a subject-specific profile such that the treatment is personalized to the individual or population. The selection may be based, for example, on a subject genotype for one or more genes related to T1DM (e.g., correlated to a subject's antigen-specific autoimmune response to preproinsulin) and/or on a subject's immune response to one or more specific peptides (e.g., as measured by a stimulation assay). By determining which one or more peptide fragments of preproinsulin a subject is likely to respond to or best respond to (e.g., which one or more peptides the subject exhibits or is likely to exhibit an autoimmune response against), a more efficient treatment may be tailored for the subject. For example, the personalized treatment may reduce cost, improve safety (e.g., reduce side-effects of administering ineffective peptide fragments), and/or improve efficacy (e.g., by allowing for increased concentrations of effective peptide fragments).

In addition to mitigating clinical T1DM, the disclosed methods and compositions can, in certain embodiments, prevent the development or progression of pre-clinical T1DM. This can be advantageous because, in various aspects and embodiments, the disclosed methods and compositions can delay the clinical onset of T1DM, thus providing a longer symptom free period, or prevent the clinical onset of T1DM altogether. At the time of diagnosis, a T1DM patient may still have appreciable amounts of insulin production (e.g., functioning beta cells as measured by C-peptide levels). An intervention that can stop or delay the loss of functional residual beta cell mass in T1DM is highly desirable because it may provide a longer ‘remission’ period after the onset of T1DM. Furthermore, the disclosed methods and compositions may reduce or delay development of acute and chronic complications in certain patients.

Similarly, the disclosed methods and compositions may significantly improve the day-to-day management for subjects with diabetes. For example, protection against hypoglycemia and provide improved metabolic control may be provided, resulting in a delay and/or reduction in the micro and macro-vascular complications of diabetes. In summary, preservation of residual beta cell function is highly desirable as it may lead to reduction of the short- and long-term complications of T1DM.

According to one embodiment, disclosed herein is a method of treating type 1 diabetes mellitus (T1DM) autoimmunity in a subject in need thereof. The method involves administering to the subject a composition comprising a selection of one or more peptide fragments of preproinsulin. The selection is based on, or based at least in part on, a genotype of the subject and/or an autoimmunity phenotype of the subject that is determined by one or more stimulation assays. The genotype and/or the autoimmunity phenotype are associated with an antigen-specific immune response to the one or more peptide fragments or to one or more preproinsulin epitopes present within the selection of the one or more peptide fragments.

According to another embodiment, disclosed herein is a method of selecting peptides suitable for treating one or more patients for type 1 diabetes mellitus (T1DM) autoimmunity. The method involves associating a selection of peptide fragments of preproinsulin to a genotype and/or an autoimmunity phenotype associated with an antigen-specific immune response to the one or more peptide fragments or to one or more preproinsulin epitopes present within the selection of the one or more peptide fragments.

The selection of the aforementioned methods may be a subset of peptide fragments from a larger set of therapeutic peptide fragments. The selection may be based on, or based at least in part on, the autoimmunity phenotype determined by the one or more stimulation assays. The one or more stimulation assays may involve exposing a plurality of peripheral blood mononuclear cells (PBMCs) to one or more stimulus peptides derived from preproinsulin. The one or more stimulus peptides may be selected from the larger set of therapeutic peptide fragments.

The autoimmunity phenotype may be a characterization of the proliferation of one or more populations of cells within the plurality of cells in response to the exposure to the one or more stimulus peptides. The one or more populations may include a population of T-cells.

The autoimmunity phenotype may be a characterization of cytokine production by one or more populations of cells within the plurality of cells in response to the exposure to the one or more stimulus peptides. The one or more populations may include a population of T cells. The characterization of cytokine production may be a characterization of the production of one or more of IFN-γ, TNF-α, TGF-β, IL-1β, IL-2, IL-4, IL-6, IL-8, IL-10, IL-12p70, IL13, and IL-17A. In specific embodiments, the characterization of cytokine production may be the characterization of the production IFN-γ. The cytokine production may be characterized by an ELISA assay. The cytokine production may be characterized by an enzyme-linked immunoassay (ELISA). The cytokine production may be characterized by an enzyme-linked immune absorbent spot (ELISpot) assay. The cytokine production may be characterized by measuring cytokine gene expression. The cytokine production may be characterized by fixing the one or more populations of cells and staining for one or more cytokines within the cells.

One or more populations of cells within the plurality of cells may be quantified by flow cytometry after the exposure to the one or more stimulus peptides. The one or more population of cells may be sorted by fluorescence-activated cell sorting (FACS). The one or more populations quantified may include one or more of the following cell types: NK cells, B cells, T-cells, naïve T-cells, memory T-cells (e.g., central memory T-cells, effector memory T-cells, and/or virtual memory T-cells), effector T-cells, helper T-cells, cytotoxic T-cells, double positive T-cells, regulatory T-cells, Th0 cells, Th1 cells, Th2 cells, and Th17 cells T-cells. In specific embodiments, the one or more populations may include two or more of the aforementioned cell types. In specific embodiments, the one or more populations may include three or more of the aforementioned cell types. The one or more populations may be labeled for one or more of the following markers: CD4, CD8, CD3, CD107a, CD25, CD40L, CD44, CD69, CD31, CD45RA, CD45RO, CD62L, CD127, CCR7, Foxp3, and γδ TCRs. In specific embodiments, the one or more populations are labeled for two or more of the aforementioned markers. In specific embodiments, the one or more populations are labeled for three or more of the aforementioned markers. The one or more populations may be labeled with one or more stimulus-specific multimers and multimer-labeled cells may be quantified for each of the one or more stimulus-specific multimers. According to some embodiments, a T-cell receptor (TCR) repertoire of one or more cells within the plurality of cells is sequenced. The one or more cells may be within one or more populations of cells sorted by FACS.

The one or more stimulus peptides may include each of the peptides of the larger set of peptide fragments. The exposure of the plurality of cells to the one or more stimulus peptides may be performed in vitro by incubating the one or more stimulus peptides with cells obtained from the subject. The exposure of the plurality of cells to the one or more stimulus peptides may be performed in vivo by administering the one or more stimulus peptides to the subject. The methods may involve performing the one or more stimulation assays. The methods may involve collecting a sample having the plurality of cells from the subject.

The autoimmunity phenotype may be a characterization determined by single cell analysis of each cell within one or more populations of cells within the plurality of cells. The single cell analysis may involve profiling for each cell one or more of a gene expression profile for one or more target genes, a protein profile for one more target proteins, a transcriptome, a B-cell receptor (BCR) clonotype, and a T-cell receptor (TCR) clonotype. In specific embodiments, the single cell analysis may involve profiling the proteome for each cell. The single cell analysis may involve labeling the cell surface receptors with specific protein binding molecules that can be detected by single cell sequencing. In specific embodiments, the single cell analysis may involve profiling the transcriptome for each cell. In specific embodiments, the autoimmunity phenotype may be a characterization of a BCR and/or TCR clonotype expansion in response to the stimulus. Characterizing the autoimmunity phenotype may involve determining the antigen-specificity of each cell within one of the one or more populations. The antigen-specificity may be determined by multimer labeling or antigen labeling. The single cell analysis may entail a multiomic analysis.

In various embodiments, the selection may be based on, or based at least in part on, the genotype of the subject. The genotype may be a genotype for one or more of an HLA gene, an insulin gene (INS gene), a protein tyrosine phosphatase non-receptor type 22 (PTPN22) gene, and a T-cell receptor (TCR) gene.

The genotype may be a genotype for an HLA-A gene, an HLA-B gene, an HLA-DRA gene, an HLA-DRB1 gene, an HLA-DQA1 gene, or an HLA-DQB1 gene. The genotype may be an HLA supertype or an HLA serotype. The genotype may be an HLA haplotype. In specific embodiments, the HLA haplotype may include a genotype for the HLA-DRB1 gene, the HLA-DQA1 gene, and/or the HLA-DQB1 gene. In specific embodiments, the genotype may characterize the presence or absence of one or more of the following haplotypes: a DRB1*04-DQA1*03:01-B1*03:02 haplotype, a DRB1*03:01-DQ A1*05:01-B1*02:01 haplotype, a DR8-DQ4 haplotype, a DR4-DQA1*03-DQB1*03:01 haplotype, a DRB1*04:05-DQB1*04:01 haplotype, and a DRB1*04:05-DQB1*04:02 haplotype. In specific embodiments, the genotype may characterize the presence or absence of any one of the haplotypes in Table 1.

The genotype may be a class I, class II, or class III categorization for the variable number of tandem repeats (VNTR) in the INS gene. The genotype may characterize the presence or absence of one or more of a L13R, A24D, R6C, and R6H mutation.

The genotype may be the genotype for amino acid position 1858 of the PTPN22 gene.

The genotype may be for the presence or absence of any one of the TCR alleles in Table 2. The genotype may be a TCR sequence. The TCR sequence may be from the aforementioned TCR repertoire sequenced for the one or more cells.

The genotype may be obtained from sequencing a blood sample or saliva sample. The genotype may be obtained from a sample used to perform at least one of the one or more stimulation assays or from a different sample. The methods may involve performing the genotyping (e.g., sequencing).

In various embodiments, the selection may be based on, or based at least in part on, the genotype and the autoimmunity phenotype.

Associating the selection of peptide fragments of preproinsulin to the subject's genotype may involve associating each of the peptide fragments within the selection to a subset of reference subjects who have demonstrated an immune response to the peptide fragment. The subset of reference subjects will share a genotype for one or more genes with the subject. The subset of reference subjects are selected from a larger group of reference subjects who were tested for an immune response against the same peptide fragment.

The group of reference subjects may have been tested for the immune response by performing a stimulation assay, such as one of the aforementioned stimulation assays. In some embodiments, the group of reference subjects may have been administered the same peptide fragment associated with the genotype, as part of a composition for treating type 1 diabetes mellitus autoimmunity, and the immune response demonstrated by the subset of reference subjects may be an immunomodulatory response. In some embodiments, the immune response is an immuno-aggressive response. At least one reference subject in the group of reference subjects may have been tested for each of the peptides from the larger set of therapeutic peptides. In specific embodiments, each reference subject was tested for each of the peptides from the larger set of therapeutic peptides. The antigen-specificity of each reference subject may be determine. In specific embodiments, the antigen-specificity is determined by using multimers to characterize or quantify antigen-specific T-cells obtained from the subject.

The method may further involve associating a second selection of peptide fragments of preproinsulin to a second subject's genotype by associating each of the peptide fragments within the second selection to a second subset of reference subjects who have demonstrated an immune response to the peptide fragment. The second subset of reference subjects will share a genotype for one or more genes with the second subject. The second subset of reference subjects are selected from the larger group of reference subjects.

According to various embodiments, the larger set of therapeutic peptide fragments is therapeutically effective to treating type 1 diabetes mellitus (T1DM) autoimmunity in a subject regardless of the subject's antigen specificity for a preproinsulin epitope. The larger set of therapeutic peptide fragments may cumulatively span at least 75%, 80%, 85%, 90%, 95%, or 99% of SEQ ID NO: 1. The spanned length may be uninterrupted. In specific embodiments, the larger set of therapeutic peptide fragments cumulatively span the entire length of SEQ ID NO: 1. Each of the peptide fragments of the larger set of therapeutic peptide fragments may be 10 to 30 amino acids in length. In specific embodiments, each of the peptide fragments of the larger set of therapeutic peptide fragments is 20 amino acids in length. Each of the peptide fragments of the larger set of therapeutic peptide fragments may have at least 85% sequence identity to the amino acid sequence of any one of SEQ ID NOs: 2-11. In specific embodiments, each of the peptide fragments of the larger set of therapeutic peptide fragments has the amino acid sequence of any one of SEQ ID NOs: 2-11. The larger set of therapeutic peptide fragments may possess two or more preproinsulin epitopes. In specific embodiments, each peptide fragment of the larger set of therapeutic peptide fragments possesses a preproinsulin epitope. The larger set of therapeutic peptide fragments may include 2, 3, 4, 5, 6, 7, 8, 9, 10, or more peptide fragments. Each peptide fragment of the larger set of therapeutic peptide fragments may overlap another of the peptide fragments. The length of each overlap may be between 5-20 amino acids. In specific embodiments, the length of each overlap is 10 amino acids. Each peptide fragment of the larger set of therapeutic peptide fragments may have an identical length and an identical length of overlap with a proximate peptide fragment. The larger set of therapeutic peptide fragments may not exhibit insulin-like metabolic activity. The selection of the one or more peptide fragments may possess a preproinsulin epitope that is not present in insulin. The selection may possess preproinsulin epitope that is not solvent accessible in insulin but which is solvent accessible in preproinsulin. The selection may possess a preproinsulin epitope spanning a junction of the signal peptide and the B chain, a junction of the B chain and the C-peptide, or a junction of the C-peptide and the A chain.

The composition may include an alum adjuvant or other pharmaceutically acceptable carrier. The composition may include an adjuvant that promotes a regulatory immune response. The composition may include an adjuvant having an oil and an emulsifier. The composition may include an incomplete Freund's adjuvant (IFA). The composition may be immunomodulatory. The composition may not be immunosuppressive. The composition may elicit a Th2 immune response. The composition may not elicit a Th1 immune response. The methods may further involve preparing the composition by combining the one or more peptide fragments of the selection, when the one or more peptide fragments comprises at least two peptide fragments.

According to another embodiment, disclosed herein is a composition according to any of the aforementioned descriptions.

According to another embodiment of the invention, disclosed herein is a kit for treating type 1 diabetes mellitus (T1DM) autoimmunity. The kit includes a therapeutically effective amount of one of the composition described above and instructions for administration of the composition to a subject in need of treatment for T1DM.

According to another embodiment of the invention, disclosed herein is another kit for treating type 1 diabetes mellitus (T1DM) autoimmunity. The kit includes a plurality of containers. Each container contains one of the peptide fragments of any one of the compositions described above, the selection of the one or more peptide fragments having at least two peptide fragments. In specific embodiments, there is a container for each peptide fragment of any one of the larger set of therapeutic peptides described above and each container contains one of the peptide fragments. The kit may include instructions for administration of the composition to a subject in need treatment for T1DM.

### DETAILED DESCRIPTION OF THE INVENTION

The present disclosure, in various aspects and embodiments, provides immunomodulatory therapy for type 1 diabetes mellitus (T1DM) autoimmunity, including therapeutics, therapies, kits, and methods for making the same. For example, the disclosure provides compositions for treating T1DM autoimmunity comprising a therapeutically effective amount of one or more peptide fragments of preproinsulin.

### References and Definitions

All publications, patent applications, patents, and other references mentioned herein are incorporated by reference herein in their entirety. The patent and scientific literature referred to herein establishes knowledge that is available to those of skill in the art. The issued US patents, allowed applications, published foreign applications, and references, which are cited herein are hereby incorporated by reference in their entirety to the same extent as if each was specifically and individually indicated to be incorporated by reference.

Aspects of the present disclosure can be embodied in different forms and should not be construed as limited to the embodiments set forth herein. Rather, these embodiments are provided so that this disclosure will be thorough and complete, and will fully convey the scope of the disclosure to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant disclosure.

Unless otherwise defined, all technical and scientific terms used herein have the same meaning as commonly understood by one of ordinary skill in the art to which this disclosure relates. The terminology used in the description herein is for the purpose of describing particular embodiments only and is not intended to be limiting unless clearly indicated otherwise by context.

As used in the specification and the appended claims, the singular forms “a,” “an” and “the” include plural referents unless the context clearly dictates otherwise. Thus, for example, reference to “a peptide fragment of preproinsulin,” “a composition” or “an additional therapeutic” can include mixtures of two or more such peptide fragment of preproinsulin, composition, or additional therapeutics, and the like.

As used herein, unless specifically indicated otherwise, the word “or” is used in the inclusive sense of “and/or” and not the exclusive sense of “either/or.” The term “and/or” encompasses embodiments in which both or either of the linked features are true or present.

The term “about” or “approximately” generally means within 10%, preferably within 5%, or more preferably within 1%, of a given value or range, unless dictated otherwise by context.

The terms “comprises”, “comprising”, “includes”, “including”, “having” and their conjugates mean “including but not limited to”.

Various embodiments of this disclosure may be presented in a range format. It should be noted that whenever a value or range of values of a parameter are recited, it is intended that values and ranges intermediate to the recited values are also part of this disclosure. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1-10 should be considered to have specifically disclosed subranges such as from 1 to 3, from 1 to 4, from 1 to 5, from 1 to 6, from 1 to 7, from 1 to 8, from 1 to 9, from 2 to 4, from 2 to 6, from 2 to 8, from 2 to 10, from 3 to 6, etc., as well as individual numbers within that range, for example, 1, 2, 3, 4, 5, 6, 7, 8, 9 and 10. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. In a particular aspect, the subject is human.

A “patient” refers to a subject who shows symptoms and/or complications of type 1 diabetes mellitus (T1DM), is diagnosed with T1DM, is under the treatment of a clinician, e.g., physician for T1DM, has pre-clinical T1DM, and/or is at a risk of developing T1DM. The term “patient” includes human and veterinary subjects. Any reference to subjects in the present disclosure, should be understood to include the possibility that the subject is a “patient” unless clearly dictated otherwise by context.

As used herein, the term “treatment” refers to the medical management of a subject, such as a patient, with the intent to cure, ameliorate, stabilize, or prevent type 1 diabetes mellitus (T1DM). This term includes active treatment (treatment directed to improve T1DM), causal treatment (treatment directed to the cause of T1DM), palliative treatment (treatment designed for the relief of symptoms or complications associated with T1DM), preventative treatment (treatment directed to delaying, minimizing, or partially or completely inhibiting the development or onset of T1DM); and supportive treatment (treatment employed to supplement another therapy). Treatment also includes curing, suppressing, reducing, alleviating, and/or ameliorating one or more symptoms and/or complications associated with T1DM. In some embodiments, treatment can include achieving at least one clinical endpoint of T1DM, such as improved C-peptide secretion, reduced insulin use, improved HbA1c, closer to normal blood sugar levels, less blood sugar level fluctuation, and the like. In some embodiments, treatment can include reducing or mitigating at least one symptom of T1DM.

For example, treatment can include reducing the frequency of hypoglycemia/hyperglycemia, reducing glucosuria, reducing a level/number of hospitalization(s), and reducing a level/number of complications such as nephropathy, neuropathy, and retinopathy. In particular, treatment can include reducing at least one symptom of T1DM by at least 5%, such as, 5%, 10%, 15%, 20%, 25%, 30%, 35%, 40%, 45%, 50%, 55%, 60%, 65%, 70%, 75%, 80%, 85%, 90%, 95%, 99% or more, as determined relative to a suitable control. A suitable control may be a similar symptom in a control subject, such as a test subject before receiving the treatment method described herein, or a different subject or group of subjects with like symptoms as the test subject, who did not receive the treatment described herein.

Treatment can also include prevention and/or delay of the onset of symptoms and/or complications associated with T1DM. Treatment also includes diminishment of the extent of T1DM; delaying or slowing the progress of the T1DM; preventing, delaying or slowing the progress of pre-clinical T1DM to clinical T1DM; preventing, delaying or slowing development of T1DM in a subject who is at a risk of developing T1DM; amelioration or palliation of T1DM; and remission (whether partial or total), whether detectable or undetectable.

“Ameliorating” or “palliating” T1DM means that the extent and/or undesirable clinical manifestations of T1DM are lessened and/or the time course of the progression is slowed or lengthened, as compared to the extent or time course in the absence of treatment. Treatment does not require the complete amelioration of a symptom, complication, or disease and encompasses embodiments in which one reduces symptoms and/or underlying risk factors.

“Treatment” can also mean prolonging survival as compared to expected survival if not receiving treatment. Those in need of treatment include those already with T1DM, as well as those prone to have the condition or disorder or those in which the condition or disorder is to be prevented. The term “prevent” does not require the 100% elimination of the possibility of an event. Rather, it denotes that the likelihood of the occurrence of the event has been reduced in the presence of the compound or method. In various aspects, the term covers any treatment of a subject, including a mammal (e.g., a human), and includes: (i) preventing T1DM from occurring in a subject that can be predisposed to the disease but has not yet been diagnosed as having it; (ii) inhibiting T1DM, such as arresting its development; or (iii) relieving T1DM, such as causing regression of the T1DM.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compositions or methods disclosed herein. In some aspects of the disclosed methods, the subject has been diagnosed with a need for treatment of T1DM prior to the administering step. As used herein, a subject in need of a treatment may refer to identification of or selection of a subject based upon need for treatment of T1DM. It is contemplated that the identification can, in one aspect, be performed by a person different from the person making the diagnosis. It is also contemplated, in a further aspect, that the administration can be performed by one who previously made the diagnosis.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intra-aural administration, intracerebral administration, rectal administration, and parenteral administration, including injectable administration such as intravenous administration, intra-arterial administration, intramuscular administration, and subcutaneous administration. Administration can be continuous or intermittent. In various aspects, a preparation can be administered therapeutically, such as administered to treat an existing disease or condition, such as T1DM. In further aspects, a preparation can be administered prophylactically, such as administered for prevention of a disease or condition, such as T1DM.

As used herein, the term “effective amount” or “amount effective” or “therapeutically effective amount” refer to an amount that is sufficient to achieve the desired result or to have an effect on an undesired condition. For example, a “therapeutically effective amount” may refer to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts.

For example, it is well within the skill of the art to start doses of a therapeutic at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a prophylactically effective amount, such as an amount effective for prevention of a disease or condition, such as T1DM.

As used herein with reference to preproinsulin peptide fragments or a composition containing the same, the term “unit dosage form” refers to the amount of the one or more preproinsulin peptide fragments and/or the composition that is suitable for administration to a subject in a single dose. In some embodiments, a unit dosage form of one or more preproinsulin peptide fragments and/or a composition (e.g., a pharmaceutical composition) described herein may encompass a therapeutically effective amount of the preproinsulin peptide fragments and/or the composition.

The term “pharmaceutically acceptable” describes a material that is not biologically or otherwise undesirable, such as without causing an unacceptable level of undesirable biological effects or interacting in a deleterious manner.

As used herein with respect to a parameter, the term “reduce” or “reducing” or “decrease” or “decreasing” or “alleviate” or “alleviating” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 97%, 99%, or more) negative change in the parameter from a comparison control, e.g., an established normal or reference level of the parameter, or an established standard control. For example, as used herein, reducing or decreasing or alleviating symptoms and/or complications associated with T1DM refers to detectable (e.g., at least about 5%, 10%, 15%, 20%, 25%, 30%, 35%, 40%, 45%, 50%, 55%, 60%, 65%, 70%, 75%, 80%, 85%, 90%, 95%, 97%, 99%, or more) negative change in symptoms and/or complications associated with T1DM in a test subject (e.g., a subject who was subject to the methods of treatment described herein) compared to symptoms and/or complications associated with T1DM in a control subject (e.g., the same subject before receiving the treatment method described herein; or a different subject, or group of subjects with like symptoms as the test subject, who did not receive the treatment described herein).

As used herein, a “control” or “control subject” refers to a subject who has not received the compositions and methods of the present disclosure. As used herein, a “test subject” refers to a subject who has received the compositions and methods of the present disclosure. As used herein with reference to a parameter, a “suitable control” may refer to the parameter in a control subject (e.g., a test subject before receiving the treatment method described herein; or a different subject, or group of subjects with like symptoms as the test subject, who did not receive the treatment described herein). For example, as used herein with reference to symptoms and/or complications associated with T1DM, a “suitable control” may refer to symptoms and/or complications associated with T1DM in a control subject (e.g., a test subject before receiving the treatment method described herein; or a different subject, or group of subjects with like symptoms as the test subject, who did not receive the treatment described herein).

As used interchangeably herein, the terms “peptide fragments” or “peptide fragments of preproinsulin” or “preproinsulin peptide fragments” refer to fragments of preproinsulin protein, e.g., human preproinsulin protein. Reference to the preproinsulin peptide fragments may refer to a collection or composition of preproinsulin peptide fragments configured for use in the one of the methods or compositions described herein, such as a therapeutic composition, and specifically to the identity (e.g., sequence) of a selection of preproinsulin peptide fragments included in the composition unless dictated otherwise by context.

As used herein, an “antigen” may refer to a molecule or molecular structure (e.g., a portion of molecule, such as a peptide fragment) that can be bound by antigen-specific receptors on various immune cells (e.g. by T-cell receptors on T-cells) or antibodies so as to induce some type of immune response. An immune response may comprise the reaction of the cells within or from the subject (e.g., any type of leukocyte) and/or the reaction of fluids within or from the subject (e.g., the reaction of humoral components within blood or lymph such as antibodies, complement proteins, and/or antimicrobial peptides) to the presence of an antigen. It will be understood that antigen-specificity may be discussed in reference to the specific molecular structure or epitope of a common antigen molecule, such as preproinsulin, that may comprise multiple specific antigens and/or epitopes.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including matters of logic with respect to arrangement of steps or operational flow; plain meaning derived from grammatical organization or punctuation; and the number or type of embodiments described in the specification.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments unless the embodiment is inoperative without those elements.

### Insulin and Preproinsulin

Autoantibodies against insulin are frequently found in newly diagnosed diabetic patients. Insulin is synthesized in the pancreatic islet B-cells from its precursor preproinsulin. Insulin is both produced and degraded within the pancreatic β-cells. Preproinsulin is a 110 amino acid biologically inactive precursor to the biologically active endocrine hormone insulin. Preproinsulin is converted into proinsulin by signal peptidases, which remove its signal peptide from its N-terminus. Finally, proinsulin is converted into the bioactive hormone insulin by removal of its connecting peptide (C-peptide).

Almost no preproinsulin exists outside B-cells because removal of the signal peptide is not a separate step, but rather is closely linked to translocation of the protein into the endoplasmic reticulum (ER). For the same reason, preproinsulin is rarely used medicinally, unlike insulin, the mature product, and proinsulin, a stable ER intermediate.

Provided herein are compositions comprising one or more peptide fragments of preproinsulin. In some embodiments, the preproinsulin is human preproinsulin (GenBank Accession No: NP_000198.1). In some embodiments, the preproinsulin is a 110 amino acid protein. The preproinsulin may comprise an amino acid sequence having at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence of SEQ ID NO: 1. For example, the preproinsulin may comprise the amino acid sequence of SEQ ID NO: 1. In those instances, a composition provided herein may contain one or more peptide fragments of SEQ ID NO: 1.

In some embodiments, the present disclosure contemplates not only SEQ ID NO: 1, but also homologs and analogs thereof. For example, a preproinsulin sequence disclosed herein can be structurally and/or functionally homologous to SEQ ID NO: 1. Homology can include at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) homology. Analogous sequences can include preproinsulin sequences from non-human species, and synthetic peptide sequences comprising one or more preproinsulin epitopes or cross-reactive epitopes. In some embodiments, analogous sequences can include human preproinsulin sequences containing one or more mutations or polymorphisms.

The first step of insulin biosynthesis involves the targeting and translocation of newly synthesized preproinsulin from the cytosol into the endoplasmic reticulum (ER). This process is led by the signal peptide of preproinsulin at its N-terminus. Preproinsulin has a 24 residue signal peptide, which comprises three regions: a positive charged n-region; a central core hydrophobic h-region; and a polar c-region containing a cleavage site of the SPase. Mutations located in the preproinsulin signal peptide that have been reported to cause diabetes include, without limitations L13R, A24D, R6C, and R6H (Liu et al., Vitam Horm, 95: 35-62 (2014); Rapoport, Nature, 450: 663-669 (2007); Liu et al., Mol Aspects Med, 42: 3-18 (2015)). The clinical diabetes phenotypes associated with these mutants range from severe neonatal-onset insulin-deficient diabetes caused by L13R or A24D, to mild adult onset diabetes associated with R6C or R6H, suggesting the possibility that different cellular defects or molecular mechanisms may underlie the onset and development of diabetes in these patients (Liu et al., Mol Aspects Med, 42: 3-18 (2015)). In some embodiments, a preproinsulin sequence of the present disclosure is a human preproinsulin sequence containing one or more of L13R, A24D, R6C, and/or R6H mutations.

Insulin biogenesis begins with the synthesis of preproinsulin in the rough ER and conversion of preproinsulin to proinsulin. Preproinsulin is converted to proinsulin shortly after (or during) translocation into the lumen of the rough ER. Proinsulin is then transported to the trans-cisternae of the Golgi complex where it is directed towards nascent, immature secretory granules. Conversion of proinsulin to insulin and C-peptide by proteolytic cleavage arises within secretory granules, and is dependent upon their acidification via ATP-dependent proton pump. The proinsulin consists of the B-chain, C-peptide and A-chain. The C-peptide is cut out and the B-chain and A-chain ends connected by disulfide bonds to form insulin. The secretory granules undergo a maturation process in which insulin content becomes crystallized with zinc and calcium as dense-core granules. These new mature dense-core insulin granules form two distinct intracellular pools, the readily releasable pools (RRP) and the reserved pool. These two populations of dense-core granules may be responsible for the biphasic nature of insulin release. The RRP granules are associated with the plasma membrane and undergo an acute calcium-dependent release responsible for first phase insulin secretion. These granule contents are discharged by exocytosis in response to an appropriate stimulus, primarily glucose. This process represents the regulated secretory pathway to which more than 99% of proinsulin is directed in beta cells of a healthy individual. In contrast, second phase insulin secretion requires the trafficking of the reserved granule pool to the plasma membrane, and involves the rapid transfer of products from the Golgi complex to the plasma membrane for immediate release. The initial trigger for insulin granule fusion with the plasma membrane is a rise in intracellular calcium and in the case of glucose stimulation results from increased production of ATP, closure of the ATP-sensitive potassium channel and cellular depolarization. In turn, this opens voltage-dependent calcium channels allowing increased influx of extracellular calcium. Calcium may bind to members of the fusion regulatory proteins synaptogamin that functionally represses the fusion inhibitory protein complex.

In brief, preproinsulin is a beta cell specific antigen and, thus, can form the basis of the immunomodulatory compositions and therapies for T1DM in accordance with the present disclosure.

### Peptide Fragments of Preproinsulin

The present disclosure, in various embodiments, utilizes preproinsulin by dividing the preproinsulin sequence, or a portion thereof, into metabolically inactive overlapping preproinsulin polypeptide fragments, to capture the immune modulatory potentials of preproinsulin as a beta cell restricted antigen. By dividing preproinsulin into overlapping peptides, the immune system can be presented with peptide sequences which comprise sequences that are unique to the preproinsulin, but that are not present either in the insulin or in the C-peptides (both of which are present in circulation).

In some embodiments, the present disclosure provides a composition comprising a therapeutically effective amount of one or more peptide fragments of preproinsulin. For example, a composition described herein may contain a therapeutically effective amount of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 peptide fragments of preproinsulin. In particular instances, a composition described herein contains a therapeutically effective amount of 10 peptide fragments of preproinsulin.

Each of the one or more peptide fragments of preproinsulin can be about 5, 6, 7, 8, 9, 10, 11, 12, 13, 14, 15, 16, 17, 18, 19, 20, 21, 22, 23, 24, 25, 26, 27, 28, 29, or 30 amino acids in length. In certain instances, each of the one or more peptide fragments of preproinsulin is about 5-10, 5-15, 5-25, 10-20, or 10-30 amino acids in length. For example, each of the one or more peptide fragments of preproinsulin can be about 20 amino acids in length. In certain instances, a composition described herein comprises two or more peptide fragments of uniform length. For example, a composition of the present disclosure may comprise one or more peptide fragments of preproinsulin, wherein each peptide fragment is 20 amino acids long. In some embodiments, compositions in accordance with the present disclosure can include fragments of uniform length (e.g., all about 20 amino acids in length) as well as distributions of different lengths. Fragment lengths, or distributions thereof, can be selected to optimize an immunomodulatory effect.

In some embodiments, the one or more preproinsulin peptide fragments can comprise an amino acid sequence having at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence of any one of SEQ ID NOs: 2-11. In certain instances, the one or more peptide fragments of preproinsulin can comprise the amino acid sequence of any one of SEQ ID NOs: 2-11. In particular, a composition of the present disclosure may comprise one or more (e.g., 1, 2, 3, 4, 5, 6, 7, 8, 9, or all) of Peptide 1, Peptide 2, Peptide 3, Peptide 4, Peptide 5, Peptide 6, Peptide 7, Peptide 8, Peptide 9, and Peptide 10 described in Table 3. Peptides 1-10 in Table 3 cumulative span the entire length of SEQ ID NO: 1, each being 20 amino acids in length, and each overlapping the preceding or following peptide (based on position within SEQ ID NO: 1), if present, by 10 amino acids.

In some embodiments, a composition of the present disclosure comprises overlapping peptide fragments of preproinsulin. For example, a composition described herein may comprise two or more peptide fragments, wherein each peptide fragment overlaps with another peptide fragment. In some instances, each overlap is an overlap of about 5, 6, 7, 8, 9, 10, 11, 12, 13, 14, 15, 16, 17, 18, 19, 20, 21, 22, 23, 24, 25, 26, 27, 28, 29, or 30 amino acids. In some instances, each overlap is an overlap of about 5-10, 5-15, 5-20, 5-25, or 5-30 amino acids. For example, each overlap can be an overlap of about 10 amino acids. In certain instances, a composition described herein comprises preproinsulin peptide fragments of uniform overlap (e.g., all about 10 amino acids) as well as varying overlap. Again, overlap lengths, or distributions thereof, can be selected to optimize an immunomodulatory effect.

The one or more peptide fragments described herein may span at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) of the preproinsulin sequence. For example, the one or more peptide fragments described herein may cumulatively span at least 75% of SEQ ID NO: 1. In some instances, the one or more peptide fragments cumulatively span the entire length of the preproinsulin sequence. Thus, a composition comprising a predetermined set of peptide fragments (e.g., one hundred fragments that are each 10 amino acids long) can encompass the entire preproinsulin sequence (e.g., 110 amino acids). In certain instances, the one or more peptide fragments span the entire length of SEQ ID NO: 1. In particular, the spanned length can be uninterrupted. In other instances, the peptide fragments do not cover the entire preproinsulin sequence, and may be limited to a set or subset of preproinsulin epitopes. The one or more peptide fragments described herein may comprise at least one internal preproinsulin epitope. An internal preproinsulin epitope is an epitope which is not normally solvent accessible in insulin. For example, an internal preproinsulin epitope may comprise an epitope which is not solvent accessible in insulin, but which is solvent accessible in preproinsulin. Additionally, an internal preproinsulin epitope may comprise an epitope which is crumpled and/or hidden inside the 3D structure of the protein but which becomes exposed during the autoimmunity process. Thus, unlike an external epitope, an internal preproinsulin epitope may not be readily available to the immune system, such as in case of an immune response that is directed against the non-denatured fully-folded protein. However, an internal preproinsulin epitope may play a major role in driving the immune response in case of autoimmunity, especially when a lot of cellular debris are produced by autoimmunity reactions.

For example, the one or more peptide fragments of preproinsulin may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more preproinsulin epitopes. In certain instances, each of the one or more peptide fragments comprises an internal preproinsulin epitope. The epitopes can be selected to optimize an immunomodulatory effect.

In some embodiments, the one or more peptide fragments of preproinsulin do not exhibit insulin-like metabolic activity (e.g., in a human subject). Such embodiments can be advantageous because they can allow for administration of concentrations of peptide fragments that are greater than a preferred, or maximum tolerated, dose of insulin. In various embodiments, the fragments comprise at least one epitope that is not present in insulin. Such embodiments can advantageously limit the effect of compositions in accordance with the present disclosure to cells containing preproinsulin.

In one embodiment, each of the overlapping peptide fragments comprises a preproinsulin epitope. Preproinsulin epitopes described herein can include known epitopes, such as B chain B9-23 and A chain 1-15 epitopes. Preproinsulin epitopes can include cryptic epitopes, which under normal conditions are not generated in sufficient amounts to be recognized by T cells undergoing deletion in thymus or anergy in the periphery. See, e.g., Lanzavecchia, Exp Med. 1995 June 1:181(6): 1945-8 (doi: 10.1084/jem.181.6.1945), which is herein incorporated by reference in its entirety. Cryptic preproinsulin epitopes may be exposed as a result of fragmenting preproinsulin (i.e., epitopes that are not solvent accessible in native, folded preproinsulin). During slicing and processing, following removal of the signal peptide sequence, the preproinsulin is broken up into the A chain, C peptide, and B chain. During this process, two amino acids on both ends of C peptide (i.e., four amino acids in total) are lost and not present in any further peptides. Accordingly, the border regions of the C peptide and some epitopes in that region are not expressed in insulin. However, these regions and/or epitopes are “new” and potentially immunogenic in a pathological disease setting (e.g., in the debris that are generated in the destruction process in autoimmune conditions). In some embodiments, one or more peptide fragments of the present disclosure comprises one or more of such cryptic preproinsulin epitopes. Preproinsulin epitopes can include epitopes which span the junction of the signal peptide and the B chain, the junction of the B chain and the C-peptide, or the junction of the C-peptide and the A chain, and which, therefore, are not present in insulin. Preproinsulin epitopes can include the full set of epitopes present in the preproinsulin sequence (or analog thereof). Epitopes can also include one or more epitopes that are unique to beta cells (i.e., the specific target of autoimmunity in T1DM). In some embodiments, the peptide fragments of the present disclosure comprise one or more of the insulin A-chain 1-15 epitope, the B-chain 9-23 epitope, the B-chain 11-27 epitope, the C-peptide C3-27 epitope, the C-peptide C13-32 epitope, and the C-peptide C13-20 epitope. The preproinsulin peptide fragments, compositions thereof, and methods of using or making, may be any of those described in U.S. Pat. App. Pub. No. US 2016/0361397 to Orban et al., published on Dec. 15, 2016, which is herein incorporated by reference in its entirety.

Without wishing to be bound by any particular theory, a loss of self-tolerance to insulin, a primary autoantigen, may unleash auto-aggressive T cells and initiate autoimmunity. Thus, destruction of insulin producing cells can start well before clinical onset of T1DM. At clinical diagnosis of some subjects, there can still be about 20-50% of self-insulin production, which can be completely destroyed over few years without medical intervention. The destruction process is T cell-mediated, and may involve CD4+ cells. However, regulatory T cells (Tregs) that are capable of suppressing the auto-aggressive T cell population may also play a critical role. Treg cells include naturally occurring CD4+CD25+ cells and antigen-induced CD4+ Th2-like regulatory cells. An imbalance between the auto-aggressive and regulatory sets of T cells may be at the core of autoimmunity. Therefore, successful interventions may be implemented by deleting the auto-aggressive cells and/or boosting the regulatory population, in order to re-establish control and create a healthy balance.

Again, without wishing to be bound by any particular theory, antigen challenge in an autoimmune setting may stimulate beneficial changes in T cell subsets (e.g., Th2 vs. Th1), in cytokine production, and/or in induction of Treg cells. In practice, antigen-specific therapeutic approaches for autoimmune diseases may use putative self-antigens that have been implicated in the disease aetiopathogenesis. Insulin is a β-cell specific major protein and is also moderately immunogenic when used alone. However, when insulin is used, there is a concern about hypoglycemia among other side effects. Thus insulin-related peptides can be a safer choice than insulin for human use because they do not necessarily have a hypoglycemic effect. In some embodiments, a composition of the present disclosure comprises one or more peptide fragments that do not have a hypoglycemic effect when administered to a subject (e.g., a human).

Prolonged peripheral presentation of self-antigens can cause low-avidity auto reactive T cells to differentiate into memory-like auto regulatory T cells that suppress both auto reactive cytotoxic T lymphocytes (CTLs) and the presentation of self-antigens, thus, protecting beta cells from further damage. The autoimmune process in T1DM selectively kills the beta cells in the pancreatic islets and do not destroy other endocrine cells like glucagon producing alpha cells. This selectivity indicates that the self-antigen, which became autoantigen, is probably restricted to the beta cell. Preproinsulin, the precursor of insulin, is the only peptide that is uniquely present in beta cells and not in any other cells. In contrast, insulin and C-peptide are secretory products, which leave the beta cells and circulate in blood. In some embodiments, a composition described herein comprises one or more peptide fragments that are present in beta cells and not in any other cells. In some instances, a composition described herein comprises one or more peptide fragments that are not present in circulation (e.g., in a human subject).

In brief, peripheral reintroduction of the primary autoantigen, e.g., preproinsulin peptide fragments in adjuvant, can induce regulatory immune response and reestablish immune tolerance in T1DM patients. If the autoimmune process can be arrested even in this late stage, beta cells can be preserved and possibly permit their regeneration. This is a unique, T1DM-specific, targeted and non-immunosuppressive approach, and is, thus, particularly well-suited for children and young adults with T1DM and for prevention in at-risk human subjects as well.

### Adjuvants

Compositions in accordance with the present disclosure can include an adjuvant that promotes a regulatory immune response (e.g., in a human subject). In some embodiments, the composition includes an adjuvant that comprises an oil and an emulsifier mixed with water. In some embodiments, the composition includes an incomplete Freund's adjuvant (IFA). In some embodiments, the composition can include an alum adjuvant, squalene, killed bacteria, toxoids, inorganic compounds, liposomes, dendrimers, nanoemulsions, and/or the like.

An IFA (commercially available, for example, as Adjuvant Montanide ICA 51 from Seppic Inc., France) typically consists of two components, an oil, and an emulsifier. IFAs can be used with antigens to elicit cell-mediated immunity and the production of antibodies of protective isotypes (IgG2a in mice and IgG1 in primates). Different types of adjuvants share similar side effects, such as a reaction at the injection site and pyrogenicity. Alum, a commonly used adjuvant for human vaccine, also may produce an appreciable granulomatous response at the injection site.

The mode of action of an incomplete Freund's adjuvant can involve non-specific as well as specific immune responses (e.g., in a human subject). IFAs can also act as an antigen vehicle and as a slow release or long-term antigen presentation device. This can be an important characteristic of IFA as prolonged peripheral presentation of self-antigens can cause low-avidity auto-reactive T cells to differentiate into memory-like auto regulatory T cells that suppress both auto-reactive CTLs and the antigen presenting cells (APCs) self-antigens presentation. The specific enhancing effect of the IFA on the antigen immunogenicity may lead to increased humoral immunity (e.g., preferentially protective antibody production; IgG1 in humans and IgG2a in mice) and to elicit specific cell-mediated immunity (e.g., Th2 type). Because of the reliability and the duration of protection, the use of autoantigen-specific immunization therapy in T1DM can be advantageous. In some instances, a composition described herein is immunomodulatory. Additionally or alternatively, the composition may not be immunosuppressive. In some instances, a composition described herein elicits a Th2 immune response (e.g., in a human subject to whom the composition is administered). Additionally or alternatively, the composition may not elicit a Th1 immune response (e.g., in a human subject to whom the composition is administered).

### Combination Therapies

Compositions in accordance with the present disclosure can include one or more therapeutics in addition to the one or more preproinsulin peptide fragments described hereinabove. The additional therapeutic can be a therapeutic for T1DM and/or another related or coexisting condition. Examples of such additional therapeutics include, without limitations, pro-regulatory leukotrienes, cytokines (e.g., IL-10, TGF beta, and the like), or other substances for promoting or enhancing regulatory responses, or restoring self-tolerance. Other examples of additional therapeutics include anti-inflammatory leukotrienes and cytokines (e.g., an IL-1 antagonist) that block autoimmune responses. Further examples of additional therapeutics include agents promoting beta cell regeneration and/or growth (e.g., Exenatide) and/or other anti-inflammatory/anti-autoimmunity agents (e.g., Vitamin D and its analogs).

The one or more additional therapeutics can be part of the composition. Alternatively, the one or more additional therapeutics can be separate to the composition. In some such instances, the one or more additional therapeutics can be administered in combination with the composition. Alternatively, the one or more additional therapeutics can be administered separate to the composition. In some such instances, the one or more additional therapeutics can be administered concurrently with the composition. Alternatively, the one or more additional therapeutics can be administered prior to administration of the composition. For example, the one or more additional therapeutics can be administered about 5 min, 10 min, 15 min, 20 min, 25 min, 30 min, 45 min, 1 h, 1.5 h, 2 h, 2.5 h, 3 h, 3.5 h, 4 h, 4.5 h, 5 h, 5.5 h, 6 h, 7 h, 8 h, 9 h, 10 h, 11h, 12 h, 18 h, 24 h, 30 h, 36 h, 42 h, 48 h, 54 h, 60 h, 72 h, 96 h, 5 days, 6 days, 1 week, 2 weeks, 3 weeks, 4 weeks, 1 month, 2 months, 3 months, 4 months, 5 months, 6 months, or 1 year prior to administration of the composition. Alternatively, the one or more additional therapeutics can be administered subsequent to administration of the composition. For example, the one or more additional therapeutics can be administered about 5 min, 10 min, 15 min, 20 min, 25 min, 30 min, 45 min, 1 h, 1.5 h, 2 h, 2.5 h, 3 h, 3.5 h, 4 h, 4.5 h, 5 h, 5.5 h, 6 h, 7 h, 8 h, 9 h, 10 h, 11h, 12 h, 18 h, 24 h, 30 h, 36 h, 42 h, 48 h, 54 h, 60 h, 72 h, 96 h, 5 days, 6 days, 1 week, 2 weeks, 3 weeks, 4 weeks, 1 month, 2 months, 3 months, 4 months, 5 months, 6 months, or 1 year subsequent to administration of the composition.

### Pharmaceutical Compositions and Kits

In some embodiments, a composition of the present disclosure is formulated as a pharmaceutical composition. In certain instances, a pharmaceutical composition contains a composition of the present disclosure and a pharmaceutically acceptable carrier. For example, a pharmaceutical composition described herein may comprise one or more peptide fragments of preproinsulin in a pharmaceutically acceptable carrier. Alternatively, a pharmaceutical composition described herein may comprise one or more peptide fragments of preproinsulin and one or more additional therapeutics in a pharmaceutically acceptable carrier.

In particular, a pharmaceutical composition described herein may comprise a therapeutically effective amount of one or more peptide fragment of preproinsulin in a pharmaceutically acceptable carrier. Alternatively, a pharmaceutical composition described herein may comprise a therapeutically effective amount of one or more peptide fragments of preproinsulin and one or more additional therapeutics in a pharmaceutically acceptable carrier. In some embodiments, a therapeutically effective amount can be 5 micrograms to 10 milligrams, 0.5 to 4.0 milligrams, or any value there between. In some embodiments, a therapeutically effective amount can be 5, 10, 25, 50, 75, 100, 125, 150, 175, 200, 225, 250, 275, 300, 350, 400, 450, 500, 600, 700, 800, or 900 micrograms, or any value there between. In some embodiments, a therapeutically effective amount can be 1.0, 1.25, 1.5, 1.75, 2.0, 2.25, 2.5, 2.75, 3.0, 3.25, 3.5, 3.75, 4.0, 4.25, 4.5, 4.75, 5.0, 5.5, 6.0, 6.5, 7.0, 7.5, 8.0, 8.5, 9.0, 9.5, or 10 milligrams, or any value there between. Additionally or alternatively, a therapeutically effective amount may be an amount that can elicit a desirable immune response in the subject (e.g., a desirable level of antigen-specific Treg cells, suppression of cytotoxic T cell function, generation of a tolerogenic response, generation of a Th2/Treg response). In further or alternative instances, a therapeutically effective amount is an amount that can achieve at least one clinical endpoint (e.g., improved C-peptide secretion, reduced insulin use, improved HbA1c, closer to normal blood sugar levels, less blood sugar level fluctuation, and the like) in the subject. Additionally or alternatively, a therapeutically effective amount may be an amount that can mitigate at least one symptom of the T1DM (e.g., frequency of hypoglycemia/hyperglycemia, reduced glucosuria, level/number of hospitalization, and level/number of complications such as nephropathy, neuropathy, and retinopathy).

A pharmaceutically acceptable carrier may refer to sterile aqueous or non-aqueous solutions, dispersions, suspensions or emulsions, as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. A composition of the present disclosure or one or more components therein (e.g., the one or more peptide fragment of preproinsulin) can be formulated with pharmaceutically acceptable carriers or diluents as well as any other known adjuvants and excipients in accordance with conventional techniques, such as those disclosed in Remington: The Science and Practice of Pharmacy, 19th Edition, Gennaro, Ed., Mack Publishing Co., Easton, Pa., 1995.

In some embodiments, a pharmaceutical composition disclosed herein may contain one or more peptide fragments of preproinsulin, a pharmaceutically acceptable carrier, and, optionally, one or more additional therapeutics, and adjuvants. A pharmaceutical composition disclosed herein may include those suitable for oral administration, rectal administration, topical administration, inhalation, and parenteral (including subcutaneous, intramuscular, and intra-arterial, intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present disclosure suitable for parenteral administration can be prepared as solutions or suspensions of the active ingredients (e.g., one or more peptide fragments of preproinsulin with or without one or more additional therapeutics) in water. A suitable surfactant can be included, such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for injectable use may include sterile aqueous solutions or dispersions. Furthermore, the compositions can be in the form of sterile powders for the extemporaneous preparation of such sterile injectable solutions or dispersions. In all cases, the final injectable form is preferably sterile and effectively fluid for easy syringability. The pharmaceutical compositions is preferably stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present disclosure can be in a form suitable for topical use, such as, for example, an aerosol, cream, ointment, lotion, dusting powder, mouth washes, gargles, and the like. Further, the compositions can be in a form suitable for use in transdermal devices. These formulations can be prepared, utilizing active ingredients (e.g., one or more peptide fragments of preproinsulin with or without one or more additional therapeutics) disclosed herein, or pharmaceutically acceptable salts thereof, via conventional processing methods. As an example, a cream or ointment is prepared by mixing hydrophilic material and water, together with about 5 wt % to about 10 wt % of the active ingredient, to produce a cream or ointment having a desired consistency.

Pharmaceutical compositions of this disclosure can be in a form suitable for rectal administration, wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations, such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or non-aqueous techniques. A tablet containing a composition of this disclosure can be prepared by compression or molding, optionally with one or more accessory ingredients or adjuvants. Compressed tablets can be prepared by compressing, in a suitable machine, the active ingredient in a free-flowing form, such as powder or granules, optionally mixed with a binder, lubricant, inert diluent, surface active or dispersing agent. Molded tablets can be made by molding in a suitable machine, a mixture of the powdered compound moistened with an inert liquid diluent.

In addition to the aforementioned carrier ingredients, the pharmaceutical formulations described hereinabove can include, as appropriate, one or more additional carrier ingredients, such as diluents, buffers, flavoring agents, binders, surface-active agents, thickeners, lubricants, preservatives (including anti-oxidants), and the like. Furthermore, other adjuvants can be included to render the formulation isotonic with the blood of the intended recipient. Compositions comprising one or more peptide fragments of preproinsulin and optionally, one or more additional therapeutics, can also be prepared in powder or liquid concentrate form.

In some embodiments, unit dosage form for the one or more peptide fragments of preproinsulin and the one or more additional therapeutics are co-formulated. In such embodiments, unit dosage form for the one or more peptide fragment of preproinsulin and unit dosage form for the one or more additional therapeutics may be co-formulated for oral administration, inhalation, topical administration, and/or parenteral administration.

In other embodiments, unit dosage form for the one or more peptide fragments of preproinsulin and unit dosage form for the one or more additional therapeutics are formulated separately. In such embodiments, unit dosage form for the one or more peptide fragments of preproinsulin may be formulated for oral administration and unit dosage form for the one or more additional therapeutics may be formulated for parental administration. Alternatively, unit dosage form for the one or more peptide fragments of preproinsulin may be formulated for parental administration and unit dosage form for the one or more additional therapeutics may be formulated for oral administration. Alternatively, unit dosage form for the one or more peptide fragments of preproinsulin may be formulated for topical administration and unit dosage form for the one or more additional therapeutics may be formulated for parental administration. Alternatively, unit dosage form for the one or more peptide fragments of preproinsulin may be formulated for parental administration and unit dosage form for the one or more additional therapeutics may be formulated for topical administration. Alternatively, unit dosage form for the one or more peptide fragments of preproinsulin may be formulated for oral administration and unit dosage form for the one or more additional therapeutics may be formulated for inhalation. Alternatively, unit dosage form for the one or more peptide fragments of preproinsulin may be formulated for inhalation and unit dosage form for the one or more additional therapeutics may be formulated for oral administration. Alternatively, unit dosage form for the one or more peptide fragments of preproinsulin may be formulated for topical administration and unit dosage form for the one or more additional therapeutics may be formulated for inhalation. Alternatively, unit dosage form for the one or more peptide fragments of preproinsulin may be formulated for inhalation and unit dosage form for the one or more additional therapeutics may be formulated for topical administration.

In some embodiments, a pharmaceutical composition described herein may be formulated to release the one or more peptide fragment of preproinsulin with or without the one or more additional therapeutics immediately upon administration or at any predetermined time period after administration using controlled or extended release formulations. Administration of the pharmaceutical composition in controlled or extended release formulations is useful where the composition, either alone or in combination, has (i) a narrow therapeutic index (e.g., the difference between the plasma concentration leading to harmful side effects or toxic reactions and the plasma concentration leading to a therapeutic effect is small; generally, the therapeutic index, Tl, is defined as the ratio of median lethal dose (LD₅₀) to median effective dose (ED₅₀)); (ii) a narrow absorption window at the site of release; or (iii) a short biological half-life, so that frequent dosing during a day is required in order to sustain a therapeutic level.

Many strategies can be pursued to obtain controlled or extended release in which the rate of release outweighs the rate of metabolism of the pharmaceutical composition. For example, controlled release can be obtained by the appropriate selection of formulation parameters and ingredients, including, e.g., appropriate controlled release compositions and coatings. Suitable formulations are known to those of skill in the art. Examples include single or multiple unit tablet or capsule compositions, oil solutions, suspensions, emulsions, microcapsules, microspheres, nanoparticles, patches, and liposomes.

The pharmaceutical compositions may be sterilized by conventional sterilization techniques, or may be sterile filtered. The resulting aqueous solutions may be packaged for use as is or lyophilized. The lyophilized preparation may be administered in powder form or combined with a sterile aqueous carrier prior to administration. The pH of the preparations typically will be between 3 and 11, more preferably between 5 and 9 or between 6 and 8, and most preferably between 7 and 8, such as 7 to 7.5. The resulting pharmaceutical compositions in solid form may, for example, be packaged in multiple single-dose units, each containing a fixed amount of one or more peptide fragment of preproinsulin, and, optionally, one or more additional therapeutics, such as in a sealed package of tablets or capsules, or in a suitable dry powder inhaler (DPI) capable of administering one or more doses.

The pharmaceutical compositions can be prepared using standard methods known in the art by mixing the active ingredient (e.g., one or more peptide fragments of preproinsulin, and, optionally, one or more additional therapeutics) having the desired degree of purity with, optionally, pharmaceutically acceptable carriers, excipients, or stabilizers (Remington's Pharmaceutical Sciences (20th edition), ed. A. Gennaro, 2000, Lippincott, Williams & Wilkins, Philadelphia, PA). Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides, and other carbohydrates, including glucose, mannose, or dextrin; chelating agents such as EDTA; sugar alcohols such as mannitol or sorbitol; salt-forming counterions such as sodium; and/or nonionic surfactants such as TWEEN™, PLURONICS™, or PEG.

Optionally, but preferably, the formulation contains a pharmaceutically acceptable salt, preferably sodium chloride, and preferably at about physiological concentrations. Optionally, the formulations of the disclosure can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the disclosure can include a pharmaceutically acceptable surfactant at a concentration of 0.005 to 0.02%.

Also provided herein is a kit for treating T1DM autoimmunity including (i) a therapeutically effective amount of a composition in accordance with the present disclosure; and (ii) instructions for administration of the composition to a subject in need thereof.

Additionally, provided herein is a kit for diagnosing and treating T1DM autoimmunity including (i) a T1DM autoimmunity diagnostic (e.g., autoantibody testing-anti-insulin IAA, anti GAD65, anti IA2-insulinoma antigen 2, anti Zn8-zinc transporter 8 antibodies, T cell biomarkers, and the like); (ii) a therapeutically effective amount of a composition described hereinabove; and (iii) instructions for diagnosing a subject and administering the composition to the subject if the subject is in need thereof.

### Methods of Manufacture

Also provided herein is a method of making a composition in accordance with the present disclosure for treating T1DM. A person skilled in the art will appreciate that the polypeptide fragments can be synthesized using known polypeptide synthetic methodologies. One illustrative example is provided in Example 1 below. In various embodiments comprising a particular selection of preproinsulin peptide fragments, as described elsewhere herein, the individual peptide fragments selected may be combined at any suitable stage of the manufacturing process as would be understood by those skilled in the art. For example, the individual peptide fragments may be combined before being incorporated into a pharmaceutical formulation or separately compositions of peptide fragments already partially or fully incorporated into a pharmaceutical composition may be combined (e.g., mixed) in appropriate amounts to form a new final pharmaceutical composition comprising the selected peptide fragments.

### Subjects

In some embodiments, the present disclosure provides a method of treatment for T1DM autoimmunity, including (i) selecting a subject in need of a treatment for T1DM autoimmunity; and (ii) administering a therapeutically effective amount of a composition described herein to the subject. Selection of a patient in need of a treatment can include physical examination by a physician and/or laboratory tests.

A subject described hereinabove can be a mammalian subject, such as a human. In certain instances, the subject is a human patient, such as a subject with T1DM or a subject who is at a risk of developing T1DM. In one embodiment, the subject is a human adult. In another embodiment, the subject is a human juvenile.

In some instances, the subject has T1DM and the treatment achieves at least one clinical endpoint (e.g., improved C-peptide secretion, reduced insulin use, improved HbA1c, closer to normal blood sugar levels, less blood sugar level fluctuation, and the like). Additionally or alternatively, the subject may have T1DM and the treatment may mitigate at least one symptom of the T1DM (e.g., frequency of hypoglycemia/hyperglycemia, reduced glucosuria, level/number of hospitalization(s), and level/number of complications such as nephropathy, neuropathy, and retinopathy).

In some embodiments, the subject has pre-clinical T1DM and the treatment prevents or delays progression to clinical T1DM. For example, compositions and methods of the present disclosure can delay progression of pre-clinical T1DM to clinical T1DM by about 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, or more.

In some embodiments, the subject is predisposed to developing T1DM and the treatment prevents or delays development of T1DM. For example, compositions and methods of the present disclosure can delay development of T1DM by about 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, or more.

Human patients who may be selected for treatment with the methods of the present disclosure can be categorized into the following groups:

(a) Patients with Newly Diagnosed T1DM

Patients in this group generally have approximately 20% residual beta cell function at the time of diagnosis (Staeva-Vieira et al., Clin. and Exp. Immunol., 148:17-31 (2007),) and comprise the group most likely to show a rapid benefit to the composition and methods of the present disclosure. In the U.S., the incidence is 30,000 to 35,000 new T1DM patients annually. As treatment with the composition is expected to be life-long, this pool of patients will, thus, expand annually by at least 30,000 in the U.S. alone, not including the projected 3% annual increase in the incidence of T1DM.

The incidence/100,000 of T1DM in adults is similar to that for children and young adults (ages 1−14=10.3; ages 15-29 years=6.8; ages 30−49=7.3), and many adults are misdiagnosed with type 2 disease due to the misconception of T1DM as a disease only of children (Mobak et al., Diabet Med, 11: 650-655 (1994); Bruno et al., Diabetes Care, 28 (11):2613-2619 (2005)). Using predictive autoantibody markers, a prospective UK study showed that 30% of younger patients diagnosed with type 2 diabetes in fact may have an underlying autoimmune component and usually progress to insulin dependence within 3 years (Turner et al., Lancet 350:1288-1293 (1997); Devendra et al., BMJ 328:750-754 (2004)). This is consistent with the estimate that 10% of persons over age 35 diagnosed with phenotypic type 2 diabetes actually have underlying autoimmune diabetes (Stenstrom et al., Diabetes 54:S68-S72 (2005); Leslie et al., Clinical Rev. 91:1654-1659 (2006)), all of who are candidates for the T1DM treatment described herein, distinct from those with latent autoimmune diabetes in adults (LADA)). This group of misclassified patients and LADA patients can also be expected to greatly benefit from treatment with the methods described herein, especially since their disease progression takes a little longer to develop. The preproinsulin specific Treg cells induced or activated by the methods described herein, like other antigen-specific Treg cells, can influence effector-autoaggressive T cells of other antigen specificity by so called “infectious tolerance” and or “bystander” effects, which in the case of LADA patients may be particularly beneficial. Correct diagnosis/identification of these patients can be accomplished by methods known in the art, e.g., by serum autoantibody assays performed according to AMA Guidelines (available from Quest Diagnostics and ARUP Labs).

(b) Patients with Established T1DM.

There were an estimated 1.8 million (all age groups) T1DM patients (excluding 10% of patients diagnosed with type 2 diabetes but having underlying LADA) in the U.S. in 2003. Although such patients have insufficient insulin production and must be maintained on insulin therapy in the face of an ongoing anti-beta islet cell autoimmune response, some possess measurable levels of beta cells even many years after diagnosis. Importantly, these patients retain the capacity for regenerating functional beta cell activity, and it has been suggested that intervention could enable repletion of beta cells, possibly to physiologically meaningful levels (Staeva-Vieira et al., (2007), supra). In the active disease state, this potential is insufficient to overcome the ongoing loss of beta cells due to the autoimmune response; however, control of the autoimmune attack on beta cells would permit pancreatic beta cell regeneration and concomitant restoration of clinically significant insulin production. As the underlying mechanism of autoimmune destruction of beta cells is the same at all stages of the disease, patients with established T1DM have the potential of benefiting from down-regulation of autoimmune response, as induced by the treatment method described herein.

In addition, the methods described herein can be used in patients who receive a transplant of islet beta cells. Such transplants, without immunosuppression, are unlikely to be successful in the presence of an ongoing autoimmune response against beta cells. In addition, for similar reasons, the methods described herein will be beneficial when used with islet cell regeneration therapies, e.g., administration of exanatide.

(c) Individuals with a High Risk of Developing T1DM.

The average risk of a child developing T1DM is 6% if either of the child's parents or siblings have the disease compared with 0.4% risk in the general population (Tillil and Kobberling, Diabetes, 36:93-99 (1987)). This represents an estimated 360,000 at risk individuals under the age of 15, and 1.3 million at risk individuals for all age groups in the US in 2007. Early intervention has been suggested as a strategy to enhance the probability of successful therapy (Staeva-Vieira et al., (2007), supra). Screening high risk individuals for antibodies to insulin (IAA), glutamic acid decarboxylase (GAD), and insulinoma associated antigen (IA-2A) provides a reliable method of predicting the development of T1DM (Leslie et al., Diabetologia, 42:3-14 (1999); Bingley, Diabetes Care, 24:398 (2001); Achenbach, Curr Diabetes Rep, 5:98-103 (2005)), which can be used to identify candidates for the treatment methods of the present disclosure to prevent, or potentially reverse, autoimmune pathology prior to significant beta cell destruction. Identification of these individuals can be accomplished using methods known in the art, e.g., by serum autoantibody assays performed according to AMA Guidelines (e.g., assays available from Quest Diagnostics and ARUP Labs).

As used herein, “T1DM” also includes LADA (latent autoimmune diabetes in adults), and subjects who can be treated using the methods described herein include those with LADA.

### Methods of Use

Further provided herein are methods for treating T1DM in a subject in need thereof, wherein administration of a composition comprising one or more preproinsulin peptide fragments to the subject generates or expands autoantigen-specific (e.g., preproinsulin-specific) CD4+ regulatory T (Treg) cells. These cells have the capacity to “home” to the pancreatic beta cells, where they release regulatory cytokines and perform other cell-to-cell regulatory functions. Thus, the methods and compositions described herein can be used to prevent the development or progression of T1DM, or prevent or delay loss of residual beta cell mass, providing a longer remission period and delaying or preventing the onset of usually progressive T1DM-related, complications at a later stage of the life. Therefore, the methods described herein comprise administration (e.g., by intravenous, intramuscular, or subcutaneous routes) of a composition comprising one or more peptide fragments of preproinsulin as described herein to a subject, in an amount sufficient to generate a response that comprises the activation, generation, and/or expansion of Treg cells specific for that autoantigen. As used herein, autoantigen-specific Treg cells may refer to preproinsulin-specific Treg cells, i.e., Treg cells that are specific to the one or more peptide fragments of preproinsulin and/or to one or more peptides exhibited thereby.

### Personalized Compositions

Different subjects may exhibit different autoimmunity responses to the same antigen. In other words, some degree of inter-individual heterogeneity in autoimmune response is generally expected for autoimmune disorders such as T1DM. For example, different subjects exhibiting an autoimmune response against the preproinsulin molecule may have autoimmune responses against different epitopes of preproinsulin and/or may be tolerant to different epitopes of preproinsulin.

In some embodiments, subjects may be treated for an autoimmune disorder, such as T1DM, by administering a composition comprising a plurality of peptides, particularly a plurality of overlapping peptides, configured to induce an immunomodulatory response (a peptide vaccine), as described elsewhere herein. By administering a plurality of peptides that cumulatively span an entire length (i.e., 100%) or at least a significant portion of the length (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%) of an antigen, such as preproinsulin, the composition can be configured to have a higher probability of accounting for one or more different epitopes that may be specific inducers of an autoimmune response in the subject, particularly if there is significant overlap between the peptides (e.g., an overlap length sufficient to encompass the length one or more epitopes). In other embodiments, subjects may be treated for an autoimmune disorder, such as T1DM, by administering a composition comprising one or more specific peptides configured to induce an immunomodulatory response against the one or more specific epitopes responsible for the autoimmune response in the specific subject. In other words, the treatment may be a form of personalized medicine configured to target specific antigens or epitopes. To design more efficacious treatments for an individual subject, the subject may be categorized by the one or more specific antigens or epitopes that elicit or are likely to elicit an immune response in the subject and/or the one or more peptides that are otherwise determined to be best suited for treatment of the subject (most likely to elicit an immunomodulatory effect in the subject for the autoimmune disease).

In various embodiments, a plurality of subsets of peptide fragments formed from a larger set of peptide fragments, may be differentially associated with one or more categories of subjects or subject types, such that each subset has been identified as being most suitable for treating one or more specific categories of subjects, depending on a characterization or profile of immune response or likely immune response shared by subjects within the category. The larger set of peptide fragments may be referred to as a “full” set of peptide fragments. The full set of peptide fragments may comprise a sufficient number of peptide fragments to induce an immunomodulatory response in a subject exhibiting an autoimmune response but for which an antigen-specific autoimmune response has not been characterized. The full set of peptide fragments may be effective for non-subject-specific treatment of T1DM in a subject. For example, the full set may comprise a plurality of peptides that span an entire length or at least a significant portion of the length (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%) of preproinsulin as described elsewhere herein. The full set may comprise a plurality of overlapping peptides having overlaps no smaller, for example, than 5, 6, 7, 8, 9, 10, 11, 12, 13, 14, 15, 16, 17, 18, 19, or 20 amino acids. In particular embodiments, the full set of peptides may comprise any of the compositions described elsewhere herein. For example, the full set may comprise one Peptide 1, Peptide 2, Peptide 3, Peptide 4, Peptide 5, Peptide 6, Peptide 7, Peptide 8, Peptide 9, and Peptide 10 described in Table 3.

In various embodiments, an appropriate subset of peptide fragments for treating a specific subject may be determined by assessing an autoimmunity phenotype of the specific subject. An autoimmunity phenotype may comprise a characterization of a subject's autoimmune response or likely autoimmune response to one or more specific antigens. The characterization may comprise a quantitative measure of a subject's immune response to a specific antigen as described elsewhere herein (e.g., with respect to various stimulation assays). The one or more specific antigens may comprise one or more specific preproinsulin antigens. The one or more specific antigens may be peptides or specific epitopes exhibited by peptides. The peptides may comprise peptide fragments of preproinsulin or otherwise be derived from preproinsulin. In some embodiments, one or more of the peptide fragments of preproinsulin are unmodified. In some embodiments, one or more of the peptide fragments of preproinsulin are modified (e.g., the sequence is altered, such as the substitution of an alanine for one of the amino acids of preproinsulin). In some embodiments, the one or more specific antigens used to characterize the autoimmunity phenotype are selected from the therapeutic polypeptide fragments that form the larger full set configured for non-subject-specific treatment of the autoimmune disease. The one or more specific antigens used to characterize the autoimmunity phenotype may comprise each of the therapeutic polypeptide fragments from a full set of therapeutic polypeptide fragments. For example, in embodiments where a full set of therapeutic polypeptide fragments comprises ten preproinsulin peptide fragments spanning the entire length of the preproinsulin sequence (e.g. Peptides 1-10 of Table 3), the subject profile may be determined based on an immune response to one or more of the ten individual preproinsulin peptide fragments. In some embodiments, the subject profile may be determined based on a response characterized for each of the individual preproinsulin peptide fragments. In some embodiments, the subject profile may be determined based on a response characterized for a subset of the individual preproinsulin peptide fragments, such as a subset comprising 2, 3, 4, or 5 peptides selected from the larger full set of peptides (e.g., a set of 10 peptides).

In various embodiments, the autoimmunity phenotype may be determined by a stimulation as described elsewhere herein. In various embodiments, an appropriate subset of peptide fragments for treating a specific subject may be determined by assessing a genotype for one or more genes of the specific subject (genotyping the subject). A genotype for the subject may be associated or correlated to an autoimmunity phenotype, for example, as determined by a study comparing the genotypes of subjects to the autoimmunity phenotypes of the same subjects (e.g., as measured by a stimulation assay). In various embodiments, an appropriate subset of peptide fragments for treating a specific subject may be determined by a combination of a genotype of the subject and an autoimmunity phenotype of the subject.

### Stimulation Assays

Stimulation assays may be used to characterize and/or measure a subject's specific immune response to one or more stimuli, such as specific antigens that elicit an autoimmune response in the subject (an auto-aggressive response). Stimulation assays may be performed on subjects having an autoimmune disease (e.g., diagnosed with the autoimmune disease), prone to an autoimmune disease (e.g., a subject suitable for prophylactic treatment), or in the stages of progressing toward an autoimmune disease, including any of the subjects or patients described elsewhere herein. In various embodiments, subject/patient profiling may be performed ex vivo on a blood sample (e.g., whole blood or plasma) obtained from the subject. Blood samples may be treated with anti-coagulants (e.g., heparinized blood). Peripheral blood mononuclear cells (PBMCs) may be extracted from blood samples via density gradient centrifugation as is known in the art. Isolated PBMCs generally comprise lymphocytes (T cells, B cells, NK cells), monocytes, and dendritic cells. PBMCs may be cryopreserved in storage media as needed. One or more various measures may be used to characterize the PBMC response to the specific stimuli (e.g., stimulus peptides). PBMCs may be expanded in culture as is needed for one or more tests. One or more various responses of PBMCs to specific stimuli may be characterized and/or measured to define an autoimmunity phenotype for the subject as described elsewhere herein. To characterize an autoimmune response of the subject to specific stimuli, one or more stimulation assays may be performed. For example, samples of the PBMCs may be incubated (e.g., overnight) with various peptides or combinations of peptides and one or more responses of the PBMCs to the different stimuli evaluated. In some embodiments, the cells are incubated with stimulating antigens for at least about 6, 12, 18, 24, 36, 48, or 72 hours or at least about 1, 2, 3, 4, 5, 6, or 7 days. Suitable media and conditions for maintaining PBMCs in culture are well known in the art.

In some embodiments, the proliferation of PBMCs in culture may be monitored or assessed in response to one or more stimuli. Some immune cells may enhance their rate of proliferation in response to stimulation by antigen-specific stimuli. Thus, incubating the PBMCs comprising antigen-specific immune cells with a specific peptide stimulus that the cells recognize (e.g., that T-cell receptors bind to) may cause the cells to proliferate faster than cells which are not incubated with a recognized peptide stimulus. Methods for measuring cellular proliferation are well known in the art. For example, the PBMCs may be incubated with a tetrazolium dye (e.g., 3-(4,5-dimethylthiazol-2-yl)-2,5-diphenyltetrazolium bromide (MTT), 2,3-bis-(2-methoxy-4-nitro-5-sulfophenyl)-2H-tetrazolium-5-carboxanilide (XTT), 3-(4,5-dimethylthiazol-2-yl)-5-(3-carboxymethoxyphenyl)-2-(4-sulfophenyl)-2H-tetrazolium (MTS), or other water-soluble tetrazolium salts (WSTs)) which is reduced by NAD(P)H-dependent oxidoreductase enzymes in the cytoplasm of cells to produce a measurable color change commensurate with the metabolic activity of the sample. The color change may be measured by a spectrophotometer. In a similar manner, the PBMCs may be incubated with a radiolabeled thymidine (TdR), such as tritiated thymidine (3H-TdR), which is incorporated into dividing cells in a manner is proportional to the amount of cell proliferation. The level of incorporation may be measured using a liquid scintillation counter. The proliferation reagent (e.g., MTT) may be incubated with the cells for at least about 1, 2, 4, 6, 8, 10, 12, 16, or 24 hours prior to detection.

In some embodiments, cytokine production in response to different stimuli may be measured directly from the supernatant of PBMCs in culture (e.g., prior to or in place of quantifying cell populations by flow cytometry, as described elsewhere herein). Concentrations of secreted cytokines may be measured, for example, by enzyme-linked immunosorbent assays (ELISAs) or enzyme-linked immune absorbent spot (ELISpot) assays as is known in the art. Cytokines such as IFN-γ, TNF-α, TGF-β, IL-1β, IL-2 IL-4, IL-6, IL-8, IL-10, IL-12p70, IL-13, and/or IL-17A may be measured. One or more of these cytokines may be produced as a result of antigen-specific cells (e.g., T-cells) recognizing the stimulus. In some embodiments, cytokine gene expression may be measured in response to different stimuli. Cells may be lysed at one or more time points to measure gene expression. Differences in expression levels may be compared for different stimuli. Cytokines may be upregulated in cell populations comprising antigen-specific immune cells that recognize the stimulus. Cytokine gene expression may be measured by quantitative real-time polymerase chain reaction (qRT-PCR) or other suitable methods as are known in the art.

In some embodiments, PBMCs may be labeled and/or otherwise prepared for flow cytometry and various types of PBMCs quantified by flow cytometry as is known in the art. In some implementations, the cells are fixed, permeabilized, and/or stained prior to performing flow cytometry. Optionally, the cells may undergo the additional step of cell sorting (i.e. fluorescence-activated cell sorting or FACS) according to methods known in the art. The total number of cells in one or more PBMC populations (e.g., T-cells NK cells, B cells) or subpopulations (e.g., naïve T-cells, memory T-cells (e.g., central memory T-cells, effector memory T-cells, and/or virtual memory T-cells), effector T-cells, helper T-cells/CD4+ T-cells, cytotoxic T-cells/CD8+ T-cells, CD4+CD8+(double positive) T-cells, regulatory T-cells, Th0 cells, Th1 cells, Th2 cells, Th17 cells, etc.) and/or the relative number of cells in one or more PBMC populations or subpopulations (e.g., the proportion of CD4+ T-cells relative to all T-cells or relative to CD4+ and CD8+ T-cells) may be characterized for each stimulus. Various populations may or may not be mutually exclusive to one another. Cells may be labeled (e.g., stained) for one or more cell surface markers that may be used to distinguish cell type or PBMC populations. By way of example, cells may be labeled for one or more of CD4, CD8, CD3, CD107a, CD25, CD40L, CD44, CD69, CD31, CD45RA, CD45RO, CD62L, CD127, CCR7, Foxp3, γδ TCRs, etc. Various suitable cell surface marker definitions of different types of PBMC populations are well known in the art, including, for example, those described in Mousset et al., Cytometry A. 2019 June; 95(6):647-654 (doi: 10.1002/cyto.a.23724); De Rosa, Methods. 2012 July:57(3):383-91 (doi: 10.1016/j.ymeth.2012.01.001); Larbi et al., Cytometry A. 2014 January; 85(1):25-35 (doi: 10.1002/cyto.a.22351); Bacher et al., Cytometry A. 2013 August:83(8):692-701 (doi: 10.1002/cyto.a.22317), each of which is herein incorporated by reference in its entirety. Cells may also be labeled (e.g., stained) for internal markers indicative of an immune response to the stimulating peptide. For example, cells may be labeled (e.g., with cytokine-specific antibodies) for cytokines such as IFN-γ, TNF-α, TFF-β, IL-1β, IL-2 IL-4, IL-6, IL-8, IL-10, IL-12p70, IL-13, and/or IL-17A, which may be produced by the cells in response to the stimulus. Cytokine production may be measured as a number or percentage of cells producing the one or more cytokines and/or as a number of events detected by a flow cytometer.

In some embodiments, antigen-specific immune cells (e.g., cytotoxic T-cells, helper T-cells, natural killer T-cells, regulator T-cells) within a PBMC population can be labeled. Antigen-specific immune cells may be labeled with multimers (e.g., tetramers). Multimers may comprise complexes of antigen presenting molecules (e.g., MHC class I molecules for cytotoxic T-cells, MHC class II molecules for helper T-cells, or CDI proteins for natural killer T-cells) and antigen molecules. The stimulus (e.g., one of the preproinsulin peptides described herein) may be used as the antigen molecule. The multimer may be labeled such that it produces a detectable signal. For example, in some embodiments, the multimer may be formed from mixing biotinylated recombinant antigen presenting molecules with fluorophore-tagged streptavidin to produce a tetramer comprising one labelled streptavidin complexed with four recombinant antigen presenting molecules. The antigen presenting molecules may be mixed with the antigen prior to complexing or after complexing. In some implementations, recombinant antigen presenting molecules may be refolded in the presence of the antigen. T-cell receptors expressed on antigen-specific T-cells within the PBMC population can bind corresponding antigen molecules when present and T-cell co-receptors can bind the antigen-presenting molecule, binding the multimer to the antigen-specific T-cell, whereas unbound multimers may be washed away. In some embodiments, antigen-specific immune cells may be labeled with monomers of antigen presenting molecules complexed with antigens. The use of multimers (e.g., tetramers) may increase the avidity of the antigen-specific immune cell for the complexes improving detection. In some embodiments, a plurality antigen-presenting molecules may be conjugated to a polymer backbone (e.g., dextran) creating multimers having a large number of antigen-presenting molecules. In some embodiments, each antigen-presenting molecule may be labeled. In some embodiments, the polymer backbone may be labeled. In various embodiments, more than one label is present on each multimer. PBMCs may be incubated with labeled multimers that have been pre-loaded with the stimulus. Antigen-specific PBMCs may be quantified (e.g., using flow cytometry) to detect multimer-labeled immune cells. Multimer-labeling may be performed in combination with labeling of cell surface markers (e.g., CD8) as described elsewhere herein. Total populations of antigen-specific immune cells and/or sub-populations of antigen-specific cell types (e.g., cytotoxic T-cells) may be quantified or otherwise characterized. By characterizing an amount or quantifying a number of antigen-specific immune cells from a subject, an antigen-specificity for the subject may be determined.

In various embodiments, the genome, transcriptome (e.g., 3′ or 5′; coding and/or non-coding), and/or proteome of one or more of the PBMCs may be sequenced. In some instances, specific genes, transcripts, and/or proteins may be targeted for sequencing. Methods for sequencing are well known in the art and may generally comprise any of the methods described elsewhere herein. Transcriptome analysis may provide information on when and where one or more genes is turned on or off in the cells. Transcriptome analysis may quantify the number of transcripts to determine the amount of gene activity (a measure of gene expression). Methods for performing transcriptome analysis are well known in the art and include, for example, DNA microarray (a hybridization-based technique) and RNA-sequencing. Any of the sequencing performed on the one or more PBMCs may be single cell sequencing. Single cell analysis may be performed on a population of cells (e.g., at least 100, 1,000, 10,000, 50,000, 100,000, 500,000, 1,000,000 cells) to create one or more libraries of cellular data (e.g., sequences). The frequencies or relative proportions of certain cellular sequences or cellular phenotypes may be used to characterize an autoimmunity phenotype, similar to the frequency or relative proportions of cell types, as described elsewhere herein. Multiomic single cell profiles (comprising data sets from multiple “omes”, such as the genome, proteome, transcriptome, etc.) may be constructed for populations of one or more cells. By way of example, multiomic profiling may be performed by CHROMIUM™ Single Cell Gene Expression and/or CHROMIUM™ Single Cell Immune Profiling single cell analysis platforms (10× GENOMICS™). Proteomic analysis may be performed, for example, to determine the levels or amounts of cytokines generated by each cell, including the cytokines disclosed elsewhere herein (e.g., as measured by gene expression) or the levels or amount of cell surface proteins, including the cell surface receptors disclosed elsewhere herein (e.g., used to sort cells via flow cytometry). Cell surface receptors may be tagged with specific protein binding molecules (e.g., antibodies or ligands) and detected via flow cytometry, as described elsewhere herein, and/or may be tagged with “barcoded” specific protein binding molecules and detected via single cell sequencing (e.g., by identifying specific sequences of oligonucleotides tagged to the specific protein binding molecules). Gene expression may also be characterized for one or more of the PBMCs. Gene expression may comprise analysis of RNA expression (e.g., as characterized by Northern blotting, microarray, real-time PCR, etc.), promoter analysis (e.g., as characterized by detecting reporter genes/promoter fusions, ChIP assay, gel shift assay, etc.), protein expression analysis (e.g., as characterized by western blot, ELISA, etc.), and/or analysis of post-translational modifications (e.g., as characterized by detecting levels of protein phosphorylation and other post-translational modifications, mass spectrometry to analyze proteins and their modifications based on their mass, etc.)Protein sequencing may generally be performed by mass spectroscopy or Edman sequencing, as is generally known in the art. Methods for single cell proteomics may include those described for example, in Kelly, Mol Cell Proteomics. 2020 November: 19(11):1739-1748 (doi: 10.1074/mcp.R120.002234), which is herein incorporated by reference in its entirety. Cell surface proteins may be profiled at the single cell level using labeled antibodies as in flow cytometry. The sequencing or other cellular analysis may be performed in combination with any one or more of the stimulation assays described herein. The sequencing or other cellular analysis may generally be performed at any stage of the stimulation assay. For example, the stimulated PBMCs may be sorted by FACS and then cells within certain populations may be sequenced.

T-cell Receptor (TCR) or B-cell Receptor (BCR) sequencing may be performed, for instance, to characterize a TCR repertoire or BCR repertoire of a specific population of T-cells or B-cells, respectively. TCR or BCR profiling may provide the cognate chain pairing of the sequenced receptors (pairing a and B chains, or light and heavy chains, respectively). TCR or BCR profiling may identify the sequences for the V(D)J segments of the chains. TCR or BCR profiling may predict one or more CDRs of the receptor (e.g., CDR3). One or more dominant TCR and/or BCR sequences may be associated with specific antigens. Multimer labeling (T-cells) or antigen labeling (B-cells) may be used to isolate antigen-specific immune cells such that the TCRs or BCRs of those cells may be sequenced. TCR and/or BCR profiling may be used to assess clonal expansion in response to a stimulus, which may be used to characterize an autoimmunity phenotype. For example, the top number (e.g., top 1, 2, 3, 4, 5, 10, 15, 20, 25, etc.) clonotypes for one or more particular stimuli may define an autoimmunity phenotype. TCR or BCR clonotyping profiles for a population of cells may be compared to (e.g., overlaid with) gene expression profiles and/or proteomic profiles for the same population.

In some implementations, modeling algorithms may be used to predict antigen-specificity of TCRs or BCRs. The model may be based, at least in part, on data conducted from previous stimulation assays. For instance, prior sequencing performed on multimer-labeled cells may be used to make predictions based on sequences for cells that were not labeled with multimers. The model may compare sequences or structural predictions based on sequences to determine which antigen a sequenced TCR or BCR is likely to bind to or at least most likely to bind to, given a finite set of antigens (e.g., a full set of peptides described herein). Predictions may be made based on the TCR or BCR sequence of naïve T-cells or B-cells (e.g., from an unstimulated blood sample) or activated T-cells or B-cells (e.g., from one or more stimulated blood samples). PBMCs or populations of T-cells or B-cells may be stimulated with one or more preproinsulin peptide fragments or with whole preproinsulin, proinsulin, or insulin before TCRs and/or BCRs are sequenced.

In various embodiments, the stimulation assays may be performed on a plurality of cell preparations prepared according to a serial dilution. Mathematical analysis of the percentage of responding/nonresponding cell cultures at each dilution can be used to estimate the responder cell frequency in the original cell population via limiting dilution analysis as is known in the art.

In various embodiments, unstimulated cells may be used as negative controls. In various embodiments, cells stimulated with one or more irrelevant peptides (i.e. peptides known not to induce an immune response) may be used as negative controls. Measurements may optionally be normalized against negative control measurements. In various embodiments, cells stimulated with a full set of peptides (e.g., configured for non-subject-specific therapy) and/or with a preproinsulin protein may be used as positive controls. Measurements may optionally be normalized against positive control measurements. In various embodiments, stimulation assay results may be normalized against a maximum value, such as the highest value obtained for any of the stimuli. In various embodiments, stimulation assay results may be normalized against a minimum value, such as the lowest value obtained for any of the stimuli.

An immune response may be characterized for each subject's response to each stimulus. The response may be quantitative. For example, the response may be characterized according to the units of the detection modality used to assess the stimulation (e.g., as a level or fluorescence intensity). The response may be characterized according to an absolute number of cells, a concentration of cells, or a proportion of cells. For example, the response could be measured as the proportion of cytotoxic T-cells (e.g., relative to all PBMCs or to all T-cells) present in a sample of PBMCs after incubation with a stimulus. According to some implementations, the responses to each stimulus may be characterized as responsive/positive or non-responsive/negative. There may or may not be an intermediate range of indeterminate or lower-level responses. Various thresholds may be used to distinguish positive and/or negative responses. For example, in some embodiments, a positive response may be defined to be a response that is at least about 1.5×, 2×, 3×, 4×, 5×, 6×, 7×, 8×, 9×, 10×, 20×, 30×, 40×, 50×, 60×, 70×, 80×, 90×, or 100× the level of a negative control response. In some embodiments, a positive response may be defined to be a response that is at least about 0.10×, 0.15×, 0.20×, 0.25×, 0.30×, 0.40×, 0.50×, 0.60×, 0.70×, 0.76×, 0.80×, 0.90×, 0.95×, 0.99×, or 1.00× the level of a positive control response. In some embodiments, an objective threshold for characterizing positive and/or negative responses, such as one determined from literature or experimentation, is predetermined for each type of stimulation assay. In various embodiments, two or more replicates may be performed for each stimulus and/or control. Response values may be averaged. According to some implementations, the responses are ranked (e.g., highest to lowest) for each stimulus for which the subject was tested. The different stimuli may be assigned an ordinal value (e.g., 1, 2, 3, etc.) corresponding to their relative ranking.

### In Vivo Stimulation

In various embodiments, a stimulation assay may be performed using an in vivo stimulus. For example, the one or more stimulus peptides may be administered to a subject in vivo as part of a pharmaceutical composition, such as those described elsewhere herein. The pharmaceutical composition may be a composition configured for inducing an immunomodulatory affect against preproinsulin as described elsewhere herein. In various embodiments, one or more blood samples may be collected after administration of the in vivo stimulus. A stimulation assay may be performed using a blood sample as described elsewhere herein. The stimulation assay may or may not include additional in vitro stimulation (e.g., by incubating PBMCs from the blood sample with one or more peptides). The stimulation assay may characterize an immune response. The immune response may be assessed for measurements of an immuno-aggressive (auto-aggressive) response and/or an immunomodulatory response (e.g., for a subject treated with a composition configured to induce an immunomodulatory response as described elsewhere herein). For example, higher levels (e.g., total amounts, concentrations, or relative proportions) of cytotoxic T-cells may be a measure indicative of an immune-aggressive response, whereas higher levels of regulatory T-cells may be a measure indicative of an immunomodulatory response. The one or more samples may be collected from the subject approximately 6 hours, 12 hours, 18 hours, 24, hours, 36 hours, 48 hours, 72 hours, 4 days, 5 days, 6 days, 7 days, 2 weeks, 3 weeks, 4 weeks or longer after administration of the in vivo stimulus. The assessment of the immune response may be performed approximately 6 hours, 12 hours, 18 hours, 24, hours, 36 hours, 48 hours, 72 hours, 4 days, 5 days, 6 days, 7 days, 2 weeks, 3 weeks, 4 weeks or longer after administration of the in vivo stimulus. In some implementations, multiple rounds of administration (e.g., 2, 3, 4, 5, 6, 7, 8, 9, or 10 rounds) of the in vivo stimulus are performed prior to collecting a sample. Measurements may be normalized and/or averaged as described elsewhere herein.

In various embodiments, a subject may be treated with one or more preproinsulin peptide fragments, such as a full set of preproinsulin fragments. The subject may be assessed for an immune response to the treatment as described elsewhere herein. Data collected from the assessment of the immune response may be used to determine the appropriate treatment of other subjects, as described elsewhere herein, and/or may be used to determine appropriate subsequent treatments for the same subject (e.g., follow-on treatments). For example, based on an immune response of the subject to the initial treatment (e.g., using multimers to evaluate levels of antigen-specific T-cells following the treatment that are specific to each of the peptide antigens administered to the subject), a subsequent treatment may comprise administration of a smaller set of preproinsulin peptide fragments. For example, only those fragments for which the subject displayed a positive immune response after the first treatment may be administered in a subsequent treatment. Additional subject-specific information may be obtained for the subject before and/or after the initial treatment. For example, the subject may be genotyped for one or more genes of interest, as described elsewhere herein, and/or demographic (e.g., age, race/ethnicity, etc.) or personal health related information (e.g., weight, body mass index, HbA1c levels, etc.) may be obtained.

### Genotyping

In various embodiments, a subject may be genotyped for one or more genes believed to be related to the autoimmune disorder (e.g., T1DM). In some aspects, the genotyping comprises performing whole genome sequencing (e.g., standard, PCR-free, linked read (i.e. synthetic long read) or long read protocols), whole exome sequencing or targeted panel sequencing (e.g., HLA typing, SNP arrays). Genotyping may be performed by any appropriate sequencing method as is known in the art, including for example, Sanger sequencing, pyrosequencing, and/or next generation sequencing (NGS). Unless dictated otherwise by context, sequencing may be performed according to any of the methods disclosed in Liu et al., J Biomed Biotechnol. 2012:2012:251364 (doi: 10.1155/2012/251364); Pareek et al., J Appl Genet. 2011 November; 52(4):413-35 (doi: 10.1007/s13353-011-0057-x); or Heather et al., Genomics. 2016 January; 107(1):1-8 (doi: 10.1016/j.ygeno.2015.11.003), each of which is herein incorporated by reference in its entirety. Sequencing may be performed on samples collected from the subject (e.g., blood, saliva, hair follicles, etc.). In some instances, sequencing may be performed on isolated populations of cells or individual cells as described elsewhere herein (e.g., for TCR or BCR sequencing). Genotyping may be performed on one or more samples collected for performing a stimulation assay or on a different sample.

The major histocompatibility complex (MHC) is a large locus on vertebrate DNA containing a set of closely linked polymorphic genes that code for cell surface proteins called MHC molecules. MHC molecules bind and present antigen (e.g., from self-proteins or from pathogens) on the cell surface for recognition by T-cells. MHC molecules mediate the interactions of leukocytes with other cells. MHC peptide presentation is highly diverse as MHC expression is polygenic and codominant and as MHC genes are highly polymorphic. In humans, the MHC region occurs on chromosome 6, between the flanking genetic markers MOG and COL11A2 (from 6p22.1 to 6p21.3 about 29 Mb to 33 Mb on the hg38 assembly), and contains 224 genes spanning 3.6 megabase pairs (3 600 000 bases). The human MHC is also called the HLA (human leukocyte antigen) complex or simply HLA. The MHC gene family is divided into three subgroups: MHC class I, MHC class II, and MHC class III. The MHC class I and MHC class II genes are highly polymorphic with at least 19,031 alleles of class I HLA and 7,183 alleles of class II HLA being deposited in the international immunogenetics (IMGT®) database.

MHC class I molecules are expressed in all nucleated cells as well as platelets and present antigens (generally 8-10 amino acids residues in length) to cytotoxic T-cells (CD8+ cells). MHC class I molecules are heterodimers having a polymorphic heavy a subunit whose gene occurs inside the MHC locus and a small invariant β2 microglobulin subunit whose gene is located outside of the MHC locus. In humans, the α subunit of three different types of classical MHC class I molecules are encoded by three different genes-HLA-A, HLA-B, and HLA-C— and the α subunit of three different types of non-classical MHC class I molecules are encoded by three different genes-HLA-E, HLA-F, and HLA-G.

MHC class II molecules are generally expressed by professional antigen-presenting cells (APCs), including macrophages, B cells, and dendritic cells (DCs), and present antigen (generally 13-18 amino acids residues in length) to helper T-cells (CD4+ cells). APCs internalize and process antigenic proteins for cell surface presentation. MHC class II molecules are heterodimers having a polymorphic a subunit and a polymorphic β subunit, both of which occur inside the MHC locus. In humans, the α subunit and β subunit of three different types of classical MHC class II molecules—HLA-DP, HLA-DQ, and HLA-DR—are each encoded by different genes, the α subunit by HLA-DPA1, HLA-DQA1, and HLA-DRA and the β subunit by HLA-DPB1, HLA-DQB1, and HLA-DRB1, HLA-DRB3, HLA-DRB4, and HLA-DRB5 (each person having at least HLA-DRB1 and being limited to at most three of the four HLA-DRB genes). The other MHC class II proteins, DM and DO, are used in the internal processing of antigens and loading of peptides onto the HLA molecules.

In various embodiments, subjects may be genotyped for one or more HLA genes. The genotyping may determine the presence or absence of specific alleles. In some embodiments, subjects may be classified by the genotype of the specific allele for a gene. The genotyping may determine a broader categorization of the one or more genes. In some embodiments, subjects may be classified by an HLA supertype and/or serotype.

For example, HLA supertypes may be defined based on the HLA molecule's specific peptide binding affinity for a particular motif of the “main” anchor in the peptide bound for antigen presentation. MHC class I molecules bind peptides via the interaction of discrete pockets within peptide-binding grooves of the MHC formed by the α1 and α2 domains of the heavy chain with the amino acid side chains of the bound peptide. Generally, the main binding energy for HLA class I molecules is provided by the interaction of residues in position 2 and the C-terminus of the peptide with the B and F binding pockets of the HLA molecule, respectively, although side chains throughout the ligand can have a positive or negative influence on binding capacity. In some embodiments, alleles for the HLA-A and/or HLA-B genes may be assigned to one of the following supertypes: A01 (B pocket specificity: small and aliphatic; F pocket specificity: aromatic and large hydrophobic), A01 A03 (B pocket specificity: small and aliphatic; F pocket specificity: aromatic and basic), A01 A24 (B pocket specificity: small, aliphatic and aromatic; F pocket specificity: aromatic and large hydrophobic), A02 (B pocket specificity: small and aliphatic; F pocket specificity: aliphatic and small hydrophobic), A03 (B pocket specificity: small and aliphatic; F pocket specificity: basic), A24 (B pocket specificity: aromatic and aliphatic; F pocket specificity: aromatic, aliphatic and hydrophobic), B07 (B pocket specificity: Proline; F pocket specificity: aromatic, aliphatic and hydrophobic), B08 (B pocket specificity: undefined; F pocket specificity: aromatic, aliphatic and hydrophobic), B27 (B pocket specificity: basic; F pocket specificity: aromatic, aliphatic, basic and hydrophobic), B44 (B pocket specificity: acidic; F pocket specificity: aromatic, aliphatic and hydrophobic), B58 (B pocket specificity: small; F pocket specificity: aromatic, aliphatic and hydrophobic), B62 (B pocket specificity: aliphatic; F pocket specificity: aromatic). Specific assignments of alleles to supertypes are known in the art, including those described in Sidney et al., BMC Immunol. 2008 Jan. 22; 9:1 (doi: 10.1186/1471-2172-9-1), which is herein incorporated by reference in its entirety.

In some embodiments, a subject genotype may be classified by a serotype, based on the ability to distinguish between HLA antigens by antibody-binding. Specific assignments of alleles to serotypes are known in the art. In various embodiments, the subject may be genotyped as having an A24, B39, DR2, DR3, DR4, DR8, DQ2 (e.g., DQ2.2, DQ2.3, DQ2.5), DQ4, DQ5.1 DQ6.3, DQ6.4, DQ8, DQ9 serotype or haplotype referred to by the same name (e.g., DQ2 may refer specifically to DQA1*05:01-B1*02:01 and DQ8 may refer specifically to DQA1*03:01-B1*03:02, in which the serotype is defined by the β chain of the HLA isotype). The subject may be genotyped as homozygous for the serotype or haplotype or heterozygous for the serotype or haplotype.

In some embodiments, the subject may be genotyped as having a protein-specific allele. For example, the subject may be classified as having an A*24:02, A*24:07, B*39:06, DRB1*03:01, DRB1*04:05, DQB1*02:01, DQA1*03:01, DQA1*05:01, DQB1*03:01, DQB1*03:02, and/or DQB1*06:02 genotype. The subject may be genotyped as homozygous or heterozygous for any of these alleles.

In some embodiments, subjects may be classified by an HLA haplotype defined by multiple genes. The genes of the haplotype may be characterized by supertypes, serotypes, alleles, or combinations thereof. The DR3-DQ2 haplotype has been associated with the risk for autoantibodies against GAD65 (GADA) and the DR4-DQ8 haplotype has been associated with the risk for autoantibodies against insulin (IAA). HLA-DR-DQ haplotypes, in general, have been associated for risk of islet autoantibodies as well as tissue transglutaminase autoantibodies ((TGA) suggesting an important role in autoimmunity. Specific combinations of HLA-DRB1, HLA-DQA1, and HLA-DQB1 genes, in particular, have been strongly correlated with T1DM. In various embodiments, the genotype of the HLA-DR and/or HLA-DQ genes may be used to predict the antigen-specificity of a subject and/or determine the appropriate treatment for a subject, as described elsewhere herein.

For example, in some instances, the subject may be genotyped as having HLA haplotype DRB1*04-DQA1*03:01-B1*03:02 (the “DR4-DQ8” haplotype). The subject may be genotyped as homozygous for the DR4-DQ8 haplotype or heterozygous for the DR4-DQ8 haplotype. In some instances, the subject may be genotyped as having HLA haplotype DRB1*03:01-DQ A1*05:01-B1*02:01 (the “DR3-DQ2” haplotype). The subject may be genotyped as homozygous for the DR3-DQ2 haplotype or heterozygous for the DR3-DQ2 haplotype. In some instances, the subject may be genotyped as having the DR2-DQ2 haplotype. The subject may be genotyped as homozygous for the DR2-DQ2 haplotype or heterozygous for the DR2-DQ2 haplotype. In some instances, the subject may be genotyped as having the DR8-DQ4 haplotype. The subject may be genotyped as homozygous for the DR8-DQ4 haplotype or heterozygous for the DR8-DQ4 haplotype. In some instances, the subject may be genotyped as having the DR4-DQA1*03-DQB1*03:01 haplotype. The subject may be genotyped as homozygous for the DR4-DQA1*03-DQB1*03:01 haplotype or heterozygous for the DR4-DQA1*03-DQB1*03:01 haplotype. In some instances, the subject may be genotyped as having the DRB1*04:05-DQB1*04:01 haplotype. The subject may be genotyped as homozygous for the DRB1*04:05-DQB1*04:01 haplotype or heterozygous for the DRB1*04:05-DQB1*04:01 haplotype. In some instances, the subject may be genotyped as having the DRB1*04:05-DQB1*04:02 haplotype. The subject may be genotyped as homozygous for the DRB1*04:05-DQB1*04:02 haplotype or heterozygous for the DRB1*04:05-DQB1*04:02 haplotype. In some instances, the subject may be genotyped as having any one of the DRB1-DQA1-DQB1 haplotypes depicted in Table 1 below or having any of the constituent alleles thereof. The subject may be genotyped as homozygous or heterozygous for the haplotype or constituent allele.

TABLE 1
Representative DRB1-DQA1-DQB1 Haplotypes

| DRB1 | DQA1 | DQB1 |
|---|---|---|
| 01:01 | 01:01 | 05:01 |
| 01:03 | 01:01 | 05:01 |
| 03:01 | 05:01 | 02:01 |
| 04:01 | 03:01 | 03:01 |
| 04:01 | 03:01 | 03:02 |
| 04:02 | 03:01 | 03:02 |
| 04:03 | 03:01 | 03:02 |
| 04:04 | 03:01 | 03:02 |
| 04:05 | 03:01 | 03:02 |
| 04:07 | 03:01 | 03:01 |
| 07:01 | 02:01 | 02:01 |
| 07:01 | 02:01 | 03:03 |
| 08:03 | 06:01 | 03:01 |
| 11:01 | 05:01 | 03:01 |
| 11:03 | 05:01 | 03:01 |
| 11:04 | 05:01 | 03:01 |
| 12:01 | 05:01 | 03:01 |
| 13:01 | 01:03 | 06:03 |
| 13:02 | 01:02 | 06:09 |
| 13:03 | 05:01 | 03:01 |
| 14:01 | 01:01 | 05:03 |
| 15:01 | 01:02 | 06:02 |
| 15:01 | 01:02 | 06:03 |

The subject may be genotyped as heterozygous for combinations of any of the supertypes, serotypes, haplotypes, or alleles disclosed herein. For example, in some instances, the subject may be genotyped as having a heterozygous DR4-DQ8/DR3-DQ2 genotype. In some instances, the subject may be genotyped as having a heterozygous DR3-DQ2/DR2-DQ2 genotype. In some instances, the subject may be genotyped as having a heterozygous DR8-DQ4/DR4-DQ8 genotype. In some instances, the subject may be genotyped as heterozygous for the DQ2/8, DQ8/8, DQ6.4/8, DQ5.1/8, DQ4/8, DQ2/2, DQ2/9, DQ6.3/8, or DQ2/6.4 genotype.

Preproinsulin is encoded by the INS gene (NCBI GeneID: 3630), located on Chromosome 11p15.5. Pre-proinsulin is processed to proinsulin and then mature insulin once the C-peptide has been cleaved. Insulin is stored in the secretory granules of the pancreatic B-cells, but it is quickly secreted as a hormone in response to increasing blood glucose levels. The promoter region of the INS gene is polymorphic for the variable number of tandem repeats (VNTR). The VNTR region can be categorized according to three classes: VNTR I contains 26-63 repeating units (e.g., 5′-ACAGGGGTGGTGGGG-3′), VNTR II 80 units, and VNTR III 140-210 units, respectively. VNTR I alleles are associated with T1DM susceptibility and VNTR II alleles are associated with protection against T1DM. The VNTR locus is extremely polymorphic, not only in size of the VNTR but also in sequence variation. In some instances, the subject may be genotyped as having a class I, class II, or class III VNTR genotype. The subject may be genotyped as homozygous for a class I, class II, or class III VNTR genotype or heterozygous for a class I, class II, or class III VNTR genotype. In some embodiments, the subject may be genotyped as having one or more of an L13R, A24D, R6C, and R6H mutation in the insulin gene. The subject may be genotyped as homozygous or heterozygous for one or more of the L13R, A24D, R6C, and R6H mutations.

The protein tyrosine phosphatase non-receptor type 22 (PTPN22) gene, located on Chromosome Ip13, encodes for LYP (a protein tyrosine phosphatase). An SNP at position 1858 has been associated with type 1A diabetes and other autoimmune disorders, particularly for homozygous TT genotypes. The polymorphism (rs201811041) which changes an arginine at position 620 to a tryptophan and has been associated with a gain of function which is believed to decrease TCR signaling. The allele may decrease negative T-cell selection within the thymus that is dependent upon strong T cell receptor activation. Baschal et al., J Autoimmun. 2008 August:31(1):1-6 (doi: 10.1016/j.jaut.2008.03.003). The subject may be genotyped for a PTPN22 allele. In some instances, the subject may have an rs201811041C>T genotype. The subject may be homozygous or heterozygous for the genotype.

T-cell receptors (TCRs) are cell surface receptors found on T-cells (e.g., cytotoxic T-cells, helper T-cells, regulatory T-cells) that recognize antigen presented in MHC molecules, binding both the antigen and MHC molecule to form a tri-molecular complex. Most TCRs are heterodimers comprising an a chain and a β chain, encoded by the TRA and TRB genes, respectively. Each chain comprises a variable domain, which binds the peptide/MHC complex, and a constant domain, proximal to the cell membrane. The variable domain of each chain comprises three complementarity-determining regions (CDRs), with CDR3 being primarily responsible for antigen recognition. TCR repertoire diversity is produced in part from random genetic recombination of gene segments: VJ recombination in the α chain and VDJ recombination in the B chain. In various embodiments, the TCR genes of the subject may be genotyped. In some embodiments, the TCR sequences for the α chain and/or β chain, or portions thereof, of an individual cell or of a plurality of cells, capturing the clonal specificity after genetic recombination, may be sequenced as described elsewhere herein. In some embodiments, the inherited germline sequence for the for the α chain and/or B chain the α chain and/or β chain, or portions thereof, may be determined using any appropriate sequencing methodology. In various embodiments, the subject may be genotyped for one or more of the polymorphisms (alleles) disclosed in Table 2, reproduce below from Pierce et al., J Diabetes Res. 2013; 2013: 737485 (doi: 10.1155/2013/737485), which is herein incorporated by reference in its entirety. In some instances, the subject may be genotyped as homozygous for the allele or heterozygous for the allele.

TABLE 2
Representative TCR Polymorphisms

| TCR | | |
|---|---|---|
| Location | Polymorphism(s) | Genes |
| N-term | N2D | TRAV9-2 |
| CDR1α | V27M, G29V, G29R, | TRAV36, TRAV12-2, TRAV8-4, |
| | N30S, P30E, P30Q, | TRAV14-1, TRAV38-1, TRAV20 |
| | N31D, Y32S | |
| CDR2α | F55S, Q56E, A57G, | TRAV12-2, TRAV1-1, TRAV8-4, |
| | V57M, S58T, T58I, | TRAV14-1, TRAV25, TRAV8-7, |
| | A59G, K59E, Q61E | TRAV26-2, TRAV38-1 |
| CDR1β | A30V, N30E | TRBV7-7, TRBV6-6 |
| CDR2β | Q55H, Q57H, V57I, | TRBV9, TRBV19, TRBV30, |
| | D58N, G60D, S60C, | TRBV15, TRBV20-1, |
| | Q60H, L61I | TRBV10-1, TRBV3-1 |
| HV4β | G84E | TRBV7-2 |

According to various aspects of the disclosure, the subject may be genotyped for any of the alleles, mutations, or haplotypes disclosed in Noble et al., Cold Spring Harb Perspect Med. 2012 January; 2(1):a007732 (doi: 10.1101/cshperspect.a007732); Kantarova et al., Physiol Res. 2007; 56(3):255-66; Pierce et al., J Diabetes Res. 2013; 2013: 737485 (doi: 10.1155/2013/737485); or Baschal et al., J Autoimmun. 2008 August; 31(1):1-6 (doi: 10.1016/j.jaut.2008.03.003), each of which is herein incorporated by reference in its entirety.

### Selecting Subject-Specific Treatments

In various embodiments, the appropriate treatment for an individual subject may be determined to be a treatment comprising the administration of a composition of peptides comprising the same peptides for which the subject displayed an autoimmune response via a stimulation assay with the peptide, as described elsewhere herein. In various embodiments, the appropriate treatment for an individual subject may be determined to be a treatment comprising the administration of a composition of peptides comprising the same peptides for which the subject displayed an immunomodulatory response to a peptide stimulus administered as part of an immunomodulatory therapy, as described elsewhere herein. The composition of peptides administered may comprise a selection of peptides consisting essentially of only those peptides for which the subject displayed an immune response when stimulated with the peptide. In other words, if the subject was tested for an immune response against ten peptides and only displayed an immune response against three of the ten peptides, then the composition administered may comprise the three peptides to which the subject responded, but not the seven to which the subject did not respond. In some embodiments, the composition administered may comprise only the peptide for which the subject displayed the strongest immune response. In some embodiments, the composition administered may comprise a selection of peptides consisting essentially of only the top 2, 3, 4, or 5 peptides for which the subject displayed the strongest immune response.

In various embodiments, the appropriate treatment for an individual subject may be determined to be a treatment comprising the administration of a composition of peptides comprising the same epitopes as the peptides for which the subject displayed an autoimmune response via a stimulation assay with the peptide, as described elsewhere herein. Each stimulus peptide may be associated with one or more epitopes. In various embodiments, the appropriate treatment for an individual subject may be determined to be a treatment comprising the administration of a composition of peptides comprising the same epitopes as the peptides for which the subject displayed an immunomodulatory response to a peptide stimulus administered as part of an immunomodulatory therapy, as described elsewhere herein. The composition administered may comprise one or more peptides having at least one of the epitopes from each stimulus peptide for which the subject displayed an immune response, more than one epitope from each stimulus peptide for which the subject displayed an immune response, or each of the epitopes from each stimulus peptide for which the subject displayed an immune response. The epitopes may comprise any known epitope (e.g., from literature) or an epitope otherwise experimentally determined to be exhibited by the peptide (e.g., by comparing immune responses across overlapping peptides). The composition of peptides administered may comprise one or more peptides exhibiting a selection of one or more epitopes which consist essentially of only those preproinsulin epitopes for which the subject was determined to display an immune response when stimulated with the epitope (e.g., a peptide exhibiting the epitope). In other words, if the subject was tested for an immune response against ten peptides, each comprising one known epitope, and only displayed an immune response against three of the ten peptides, then the composition administered may comprise the three known epitopes of the three peptides to which the subject responded, but not the seven known epitopes of the seven peptides to which the subject did not respond. In some embodiments, the composition administered may comprise a selection of epitopes consisting essentially of only the epitope(s) of the peptide for which the subject displayed the strongest immune response. In some embodiments, the composition administered may comprise a selection of epitopes consisting essentially of only the epitopes of the top 2, 3, 4, or 5 peptides for which the subject displayed the strongest immune response.

In various embodiments, a phenotype model may be developed for correlating or associating one or more measures of an autoimmunity phenotype to an immune response for one or more stimuli and/or to an appropriate (e.g., antigen-specific) treatment. The phenotype model may be similar to a genetic model as described elsewhere herein. The phenotype model may comprise multiple measures of an autoimmunity phenotype. The phenotype model may be used to calculate a risk score as is known in the art. For example, a risk score for a subject's immune response to a particular antigen/stimulus may be calculated as a weighted sum of one or more autoimmunity phenotype measures. The phenotype measures may be a quantitative value such as any of the quantitative values that may be obtained with respect to a stimulation assay, as described elsewhere herein. The quantitative values may or may not be normalized (e.g., as described elsewhere herein) prior to weighting. In some instances, quantitative values may be assigned to immune responses. For example, a value of 1 may be assigned to a positive immune response to a particular stimulus and a value of 0 may be assigned to a negative immune response to a particular stimulus. In some instances, an immune response may be classified as one of three or more levels and equivalent numerical values may be assigned (e.g., 1, 2, or 3) based on the level of response. In some instances, values corresponding to an ordinal ranking (e.g., the relative size of a particular immune cell population, such as first, second, or third largest population) may be assigned. The risk score may be correlated to a likelihood of the subject exhibiting a particular type of immune response (e.g., a quantitative or qualitative measure as described with respect to stimulation assays) in response to a particular stimulus. The phenotype measures for calculating the risk score for a particular stimulus may comprise autoimmunity phenotype measures for only that particular stimulus or for a plurality of stimuli. The phenotype model may be trained and/or validated with data from a plurality of subjects. In some implementations, the phenotype model may be refined by machine learning as is known in the art (for example, a machine learning algorithm may be used to adjust the weights assigned to different autoimmunity phenotypes).

In various embodiments, the appropriate treatment for an individual subject may be determined based on or based at least in part on the subject's genotype for one or more genes. The genotype may refer to the presence or absence of a particular mutation or single-nucleotide polymorphism (SNP) at a particular locus within the gene or generally to the presence or absence of an allele for a particular gene. Determinations based on genotype may be independent of whether the subject is heterozygous or homozygous for the mutation, SNP, or allele or may be dependent on whether the subject is heterozygous or homozygous for the mutation, SNP, or allele. The one or more genes may be selected from a plurality of genes of interest. The genes of interest may comprise any gene that displays a correlation with an antigen-specific autoimmune and/or immunomodulatory response against preproinsulin. The genes of interest may comprise any of the genes described elsewhere herein. The genotype may comprise inherited germline genotypes and/or genotypes for specific populations of cells or individual cells (e.g., TCR or BCR sequences). The appropriate treatment may be determined based on at least 1 gene of interest, at least 2 genes of interest, at least 3 genes of interest, or more genes.

In some embodiments, the appropriate treatment for an individual subject may be determined to be a treatment that has been determined to be appropriate for one or more reference subjects having the same genotype as the subject for one or more genes. For instance, treatments comprising a subset of preproinsulin peptide fragments may be predetermined to be appropriate for subjects having one or more genotypes or combinations of genotypes. A second subset of preproinsulin peptide fragments may be predetermined to be appropriate for subjects having one or more different genotypes or different combinations of genotypes. The genotypes or combinations of genotypes associated with each treatment may or may not be mutually exclusive. In some embodiments, the appropriate treatment for the one or more reference subjects may be determined based on any of the characterizations of immune response described elsewhere herein, including for example by stimulation assays. The appropriate treatment for the one of more reference subjects may be determined by a phenotype model as described elsewhere herein.

In some embodiments, a genetic model may be developed for correlating or associating one or more genotypes to an immune response for one or more stimuli and/or to an appropriate (e.g., antigen specific) treatment. The genetic model may be a polygenic model. The genetic model may be used to calculate a polygenic risk score (PRS) as is known in the art. For example, a PRS for a subject's immune response to a particular antigen/stimulus may be calculated as a weighted sum of the subject's genotype for one or more markers, wherein the genotype is equated to some numeric value depending on the absence or presence of a mutation, SNP, or allele (e.g., 0 or 1; 0, 0.5 or 1; etc.). The PRS may be correlated to a likelihood of the subject exhibiting a particular type of immune response (e.g., a quantitative or qualitative measure as described with respect to stimulation assays) in response to a particular stimulus. The genetic model may be trained and/or validated with data from a plurality of subjects. In some implementations, the genetic model may be refined by machine learning as is known in the art (for example, a machine learning algorithm may be used to adjust the weights assigned to different genotypes).

In various embodiments, a hybrid autoimmunity phenotype/genetic model may be developed for correlating or associating one or more measures of autoimmunity phenotype and one or more genotypes to a predicted immune response for one or more stimuli and/or to an appropriate (e.g., antigen specific) treatment. The model may, for example, comprise a weighted some of one or more autoimmunity measures, as described elsewhere herein, and one or more genotypes, as described elsewhere herein.

In various embodiments, a mathematical model is used to calculate a risk score correlated to a likelihood of an immune response for one or more stimuli and/or to an appropriate (e.g., antigen-specific) treatment. In some implementations, a risk score is calculated for a plurality of available therapeutic peptides. In some embodiments, the appropriate treatment is determined to be a composition comprising only the therapeutic peptides having the highest 1, 2, 3, 4, or 5 risk scores. In some embodiments, the appropriate treatment is determined to be a composition comprising each therapeutic peptide having a risk score greater than or equal to a threshold level. The threshold level may be determined, for example, by comparing risk scores for specific peptides to functional measurements or clinical outcomes in patients treated with those specific peptides. In some embodiments, the appropriate treatment is determined to be a composition comprising only the therapeutic peptides having the highest 1, 2, 3, 4, or 5 risk scores greater than or equal to a threshold level. In some embodiments, models may be configured to calculate single risk scores for specific combinations of therapeutic peptides, such as specific combinations of 2, 3, 4, 5 or more available preproinsulin peptide fragments.

The strength of a correlation between a risk score or gene and one or more stimuli and/or between a risk score or gene and an appropriate (e.g., antigen-specific) treatment may be evaluated by any appropriate statistical means. Various measures for performing statistical analyses on correlations between input values and output values/outcomes are well known in the art. For instance, a receiver operating characteristic (ROC) curve may be used to assess the prognostic capabilities of a binary classifier (e.g., using a risk score to determine whether or not a therapeutic peptide should be included in a therapeutic composition). In various embodiments, a binary classifier used in one of the methods described herein may have an area under the curve (AUC) of at least about 0.6, 0.65, 0.70, 0.75, 0.8, 0.85, 0.90, 0.95, or higher. In various embodiments, a threshold used by one of the methods described herein may result in a sensitivity (i.e. true positive rate) of at least about 0.50, 0.55, 0.60, 0.65, 0.70, 0.75, 0.80, 0.85, 0.90, 0.95, 0.96, 0.97, 0.98, 0.99, or higher. In various embodiments, a threshold used by one of the methods described herein may result in a specificity (i.e. true negative rate) of at least about 0.50, 0.55, 0.60, 0.65, 0.70, 0.75, 0.80, 0.85, 0.90, 0.95, 0.96, 0.97, 0.98, 0.99, or higher. In various embodiments, a threshold used by one of the methods described herein may result in an odds ratio of at least about 1.25, 1.50, 1.75, 2.00, 3.00, 4.00, 5.00, 10.00 or higher, or the corresponding inverse.

### EXAMPLES

The following examples have been included to illustrate aspects of the inventions disclosed herein. In light of the present disclosure and the general level of skill in the art, those of skill appreciate that the following examples are intended to be exemplary only and that numerous changes, modifications, and alterations may be employed without departing from the scope of the disclosure.

### Example 1

### Polypeptide Synthesis

Overlapping preproinsulin 20 amino acid peptide fragments are designed such that each of the peptide fragments overlaps by 10 amino acids with the preceding peptide sequence. These peptides are made as monocomponent HPLC (C18 column) purified peptides, synthesized in a protein-core laboratory on a PROTEIN SYNTHESIZER MODEL 433A from Applied Biosystems, using amino acid preparations from Peptide International. This is a standard solid-phase peptide synthesis (SPPS) procedure, which has the following main steps:

### Chain Assembly

The assembly strategy used in the protein synthesis is ABI (Applies Biosystem Inc.)-Fmoc/Thr. The Fmoc group protects the α-amino group of the amino acid. The peptide is assembled from the C-terminus towards the N-terminus with the α-carboxyl group of the starting amino acid attached to a solid support (resin). The resin used for assembly is polystyrene bead, an insoluble support with size of 400-1000 micron in diameter swelled after washing with NMP (N-methylpyrrolidone). The resin is preloaded with the first amino acid (Thr) from the C-terminus.

The first step in chain assembly is deprotection, or removal of the protecting group. The Fmoc protecting group is removed using 22% piperidine. Conductimetric feedback of carbamate salt formed via removal of Fmoc group with piperidine/NMP can be used to show the coupling efficacy.

After deprotection, the next amino acid is activated and coupled to the deprotected amino end of the growing peptide and forms the peptide bond. Activation of the incoming amino acid carboxyl group is achieved using HBTU/HOBt.

Between couplings, the column is washed with methanol and NMP (N-methylpyrrolidone), which swells the resin and washes out residues. The cycle is repeated until a peptide of a desired length is achieved.

Then the resin is washed with DCM (dichloromethane), which removes NMP from the resin, followed by thoroughly washing the resin with highly volatile methanol, which is an easily removable solvent, and evaporation/drying.

Cleavage from the Resin and Removal of Side Chain Protecting Groups

A cleavage mixture is prepared (0.75 g crystalline phenol+0.25 g ethanedithiol+0.5 ml thioanisol+0.5 ml deionized H2O+10 ml trifluoroaceticacid). The dried peptide-resin is incubated in cool flask in ice bath (10 ml mixture/100-150 mg peptide-resin) for 1.5 h. Then the peptide is isolated from the reaction mixture by glass funnel filtration under high vacuum. The peptide is then precipitated with cold methyl t-butyl ether (MTBE) and vacuum dried.

### Purification Under Sterile Conditions

This step is performed with reverse phase HPLC. Buffer A=0.1% trifluoroaceticacid (TFA) and buffer B=70% acetonitrile, 30% H2O, 0.09% trifluoroaceticacid (TFA). By using C18 column, the elution of the sample is based upon hydrophobicity (hydrophilic sample elute earlier). The peak detection is performed by absorbance measurement of peptide bond at 214 nm and identified by mass spectrometry. The desired fraction is pooled in sterile vials and lyophilized, with a sample taken for AAA (amino acid analysis) analytical rpHPLC and Mass Spectrometry to confirm the sequence.

### Example 2

### Compositions Containing Preproinsulin Peptide Fragments

A. A composition containing preproinsulin peptide fragments is a combination of water-soluble, 20-amino acid long, overlapping, preproinsulin peptide fragments and incomplete Freund's adjuvant solution. The injections/emulsions (the final drug products) are prepared immediately before administration in a lamina-flow protected hood, under sterile condition by using high-pressure sterile syringes as a 50/50 (w/w) emulsion of human preproinsulin peptides mix solution (0.5 ml) by mixing with Montanide ISA51 (0.5 ml) (Seppic Inc.).

B. A composition containing preproinsulin peptide fragments is a combination of water-soluble, 20-amino acid long, overlapping, preproinsulin peptide fragments and incomplete Freund's adjuvant solution. The injections/emulsions (e.g., the final drug product) are pre-prepared (e.g., in a manufacturing setting) and can have an extended expanded shelf life (e.g., years).

C. A composition containing preproinsulin peptide fragments is a combination of water-soluble, 20-amino acid long, overlapping, preproinsulin peptide fragments and incomplete Freund's adjuvant solution. The injections/emulsions (e.g., the final drug products) are prepared as a kit; the two main components (e.g., peptide fragments and adjuvant) in different sealed compartments with a built in mechanism to prepare a fresh mix to be used within short period of time (e.g., days/weeks).

D. A composition containing preproinsulin peptide fragments is a combination of water-soluble, 20-amino acid long, overlapping, preproinsulin peptide fragments and incomplete Freund's adjuvant solution, where incomplete Freund's adjuvant solution is other than Montanide ISA51.

E. A composition containing preproinsulin peptide fragments is a combination of water-soluble, 20-amino acid long, overlapping, preproinsulin peptide fragments and an immunological adjuvant other than incomplete Freund's adjuvant solution (e.g., squalene; killed bacteria and toxoids; aluminum salts-alum/inorganic compounds etc. or liposomes, lipid based nanoparticles, nanoemulsion, nanogels, dendrimers or the like).

### Example 3

Therapy with Preproinsulin Peptide Fragments

A. Administer a composition in accordance with the present disclosure to a subject (e.g., of any age and/or any disease duration) who has been diagnosed with type 1 diabetes mellitus (T1DM) (e.g., a clinical diagnosis, and at least one positive T1DM-specific autoantibodies, such as IAA, GAD65, Ia2, Zn transporter8 or T1DM-specific T cell marker positive).

B. Administer a composition in accordance with the present disclosure to a subject who does not have clinical diagnosis of T1DM, but has at least one positive T1DM-specific autoantibodies (e.g., IAA, GAD65, Ia2, Zn transporter8) or T1DM-specific T cell marker. The subject can have normal glucose status or impaired glucose tolerance tested by oral glucose tolerance test. Such subjects can be identified by family screening of patients with T1DM, or by screening a larger population.

The section headings used herein are for organizational purposes only and are not to be construed as limiting the subject matter described in any way.

While the applicant's teachings are described in conjunction with various embodiments, it is not intended that the applicant's teachings be limited to such embodiments. On the contrary, the applicant's teachings encompass various alternatives, modifications, and equivalents, as will be appreciated by those of skill in the art.

TABLE 3
Sequence Summary Table

| SEQ | | |
|---|---|---|
| ID NO | Description | Sequence (NH2-COOH) |
| 1 | Human preproinsulin | MALWMRLLPLLALLALWGPDPAAAFVNQHL |
| | (NP_000198.1) | CGSHLVEALYLVCGERGFFYTPKTRREAED |
| | | LQVGQVELGGGPGAGSLQPLALEGSLQKRG |
| | | IVEQCCTSICSLYQLENYCN |
| 2 | Peptide 1 | MALWMRLLPLLALLALWGPD |
| 3 | Peptide 2 | LALLALWGPDPAAAFVNQHL |
| 4 | Peptide 3 | PAAAFVNQHLCGSHLVEALY |
| 5 | Peptide 4 | CGSHLVEALYLVCGERGFFY |
| 6 | Peptide 5 | LVCGERGFFYTPKTRREAED |
| 7 | Peptide 6 | TPKTRREAEDLQVGQVELGG |
| 8 | Peptide 7 | LQVGQVELGGGPGAGSLQPL |
| 9 | Peptide 8 | GPGAGSLQPLALEGSLQKRG |
| 10 | Peptide 9 | ALEGSLQKRGIVEQCCTSIC |
| 11 | Peptide 10 | IVEQCCTSICSLYQLENYCN |
| 12 | Preproinsulin-Signal Peptide | MALWMRLLPLLALLALWGPDPAAA |
| 13 | Preproinsulin-B chain | FVNQHLCGSHLVEALYLVCGERGFFYTPKT |
| 14 | Preproinsulin-C peptide | EAEDLQVGQVELGGGPGAGSLQPLALEGSLQ |
| 15 | Preproinsulin-A chain | GIVEQCCTSICSLYQLENYCN |

1. A method of treating type 1 diabetes mellitus (T1DM) autoimmunity in a subject in need thereof, the method comprising administering to the subject a composition comprising a selection of one or more peptide fragments of preproinsulin,
wherein the selection is based on or based at least in part on a genotype of the subject and/or an autoimmunity phenotype of the subject determined by one or more stimulation assays, the genotype and/or the autoimmunity phenotype being associated with an antigen-specific immune response to the one or more peptide fragments or to one or more preproinsulin epitopes present within the selection.

2. A method of selecting peptides suitable for treating one or more patients for type 1 diabetes mellitus (T1DM) autoimmunity, the method comprising:
associating a selection of peptide fragments of preproinsulin to a genotype and/or an autoimmunity phenotype associated with an antigen-specific immune response to the one or more peptide fragments or to one or more preproinsulin epitopes present within the selection.

3. The method of claim 1, wherein the selection is a subset of peptide fragments from a larger set of therapeutic peptide fragments.

4. The method of claim 1 wherein the selection is based on or based at least in part on the autoimmunity phenotype determined by the one or more stimulation assays.

5. The method of claim 4, wherein the one or more stimulation assays comprise exposing a plurality of cells comprising peripheral blood mononuclear cells (PBMCs) to one or more stimulus peptides derived from preproinsulin, optionally wherein the one or more stimulus peptides are selected from the larger set of therapeutic peptide fragments.

6. The method of claim 5, wherein the autoimmunity phenotype comprises a characterization of the proliferation of one or more populations of cells within the plurality of cells in response to the exposure to the one or more stimulus peptides, optionally wherein the one or more populations comprises a population of T-cells.

7. The method of claim 6, wherein the autoimmunity phenotype comprises a characterization of cytokine production by one or more populations of cells within the plurality of cells in response to the exposure to the one or more stimulus peptides, optionally wherein the one or more populations comprises a population of T cells.

8. The method of claim 7, wherein the characterization of cytokine production comprises a characterization of one or more of IFN-γ, TNF-α, TGF-β, IL-1β, IL-2, IL-4, IL-6, IL-8, IL-10, IL-12p70, IL13, and IL-17A.

9. The method of claim 8, wherein the characterization of cytokine production comprises the characterization of IFN-γ.

10. The method of claim 7, wherein the cytokine production is characterized by an ELISA assay.

11. The method of claim 8, wherein the cytokine production is characterized by an enzyme-linked immunoassay (ELISA).

12. The method of claim 7, wherein the cytokine production is characterized by an enzyme-linked immune absorbent spot (ELISpot) assay.

13. The method of claim 7, wherein the cytokine production is characterized by measuring cytokine gene expression.

14. The method of claim 7, wherein the cytokine production is characterized by fixing the one or more populations of cells and staining for one or more cytokines within the cells.

15. The method of claim 6, wherein one or more populations of cells within the plurality of cells are quantified by flow cytometry after the exposure to the one or more stimulus peptides, optionally wherein the one or more population of cells are sorted by fluorescence-activated cell sorting (FACS).

16. The method of claim 15, wherein the one or more populations comprise one or more of the following cell types: NK cells, B cells, T-cells, naïve T-cells, memory T-cells (e.g., central memory T-cells, effector memory T-cells, and/or virtual memory T-cells), effector T-cells, helper T-cells, cytotoxic T-cells, double positive T-cells, regulatory T-cells, Th0 cells, Th1 cells, Th2 cells, and Th17 cells T-cells.

17. (canceled)

18. (canceled)

19. The method of claim 15, wherein the one or more populations are labeled for one or more of the following markers: CD4, CD8, CD3, CD107a, CD25, CD40L, CD44, CD69, CD31, CD45RA, CD45RO, CD62L, CD127, CCR7, Foxp3, and γδ TCRs.

20-85. (canceled)

86. The composition of claim 1.

87. A kit for treating type 1 diabetes mellitus (T1DM) autoimmunity comprising:
a therapeutically effective amount of the composition of claim 86; and
instructions for administration of the composition to a subject in need thereof.

88. A kit for treating type 1 diabetes mellitus (T1DM) autoimmunity comprising:
a plurality of containers, each container comprising one of the peptide fragments of the composition of claim 86, wherein the selection comprises at least two peptide fragments, optionally wherein each container comprises one of the peptide fragments of the larger set of therapeutic peptides of claim 3; and
optionally, instructions for administration of the composition to a subject in need thereof.


SEQUENCE LISTING (15 sequences)

SEQ ID NO: 1 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 20 aa, Homo sapiens
MALWMRLLPLLALLALWGPD

SEQ ID NO: 3 - protein, 20 aa, Homo sapiens
LALLALWGPDPAAAFVNQHL

SEQ ID NO: 4 - protein, 20 aa, Homo sapiens
PAAAFVNQHLCGSHLVEALY

SEQ ID NO: 5 - protein, 20 aa, Homo sapiens
CGSHLVEALYLVCGERGFFY

SEQ ID NO: 6 - protein, 20 aa, Homo sapiens
LVCGERGFFYTPKTRREAED

SEQ ID NO: 7 - protein, 20 aa, Homo sapiens
TPKTRREAEDLQVGQVELGG

SEQ ID NO: 8 - protein, 20 aa, Homo sapiens
LQVGQVELGGGPGAGSLQPL

SEQ ID NO: 9 - protein, 20 aa, Homo sapiens
GPGAGSLQPLALEGSLQKRG

SEQ ID NO: 10 - protein, 20 aa, Homo sapiens
ALEGSLQKRGIVEQCCTSIC

SEQ ID NO: 11 - protein, 20 aa, Homo sapiens
IVEQCCTSICSLYQLENYCN

SEQ ID NO: 12 - protein, 24 aa, Homo sapiens
MALWMRLLPLLALLALWGPDPAAA

SEQ ID NO: 13 - protein, 30 aa, Homo sapiens
FVNQHLCGSHLVEALYLVCGERGFFYTPKT

SEQ ID NO: 14 - protein, 31 aa, Homo sapiens
EAEDLQVGQVELGGGPGAGSLQPLALEGSLQ

SEQ ID NO: 15 - protein, 21 aa, Homo sapiens
GIVEQCCTSICSLYQLENYCN